SEQUENCE LISTING (329 sequences)

SEQ ID NO: 1 - DNA, 48 bp, Homo sapiens
gaggattctattttttggcgtggttggtattggggtgcttttgatgtt

SEQ ID NO: 2 - DNA, 36 bp, Homo sapiens
gatcatgaggttatgcatactatgggttttgatgtt

SEQ ID NO: 3 - DNA, 21 bp, Homo sapiens
tattttatgggttatgatcat

SEQ ID NO: 4 - DNA, 30 bp, Homo sapiens
atgtttctttattatgatttttttgattat

SEQ ID NO: 5 - DNA, 30 bp, Homo sapiens
tatggtcagcagtattttggttatgcttat

SEQ ID NO: 6 - DNA, 27 bp, Homo sapiens
cttgatgcttatactggttttgatcat

SEQ ID NO: 7 - DNA, 41 bp, Homo sapiens
tggttttattatcttcatcctctttttaatggttattttga

SEQ ID NO: 8 - DNA, 24 bp, Homo sapiens
gattatatgtatgattttgatgtt

SEQ ID NO: 9 - DNA, 36 bp, Homo sapiens
ggtcatatttatgatcctggttctaattttgattat

SEQ ID NO: 10 - DNA, 45 bp, Homo sapiens
atttattttaatgatactgattctgttcttcatgatgatgcttct

SEQ ID NO: 11 - DNA, 18 bp, Homo sapiens
tatggttcttttgataat

SEQ ID NO: 12 - DNA, 54 bp, Homo sapiens
tctatgtatgtttattctccttcttatatgtatgtttcttataaggatgatcat

SEQ ID NO: 13 - DNA, 33 bp, Homo sapiens
gctattaattgggatggttctattcttgatttt

SEQ ID NO: 14 - DNA, 27 bp, Homo sapiens
attgattattattatggtgatgattat

SEQ ID NO: 15 - DNA, 26 bp, Homo sapiens
catccttttcagacttatggttatta

SEQ ID NO: 16 - DNA, 33 bp, Homo sapiens
gatggtttttatcttggtgttcctcttgattct

SEQ ID NO: 17 - DNA, 60 bp, Homo sapiens
tgggtgagcggtatctctggttcttctagccttacctcttatgcggatagcgtgaaaggc

SEQ ID NO: 18 - DNA, 60 bp, Homo sapiens
tgggtgagcaatatctctggttctggtagcaatacctattatgcggatagcgtgaaaggc

SEQ ID NO: 19 - DNA, 59 bp, Homo sapiens
tggatgggcggtatcattccggagtttggcactgcgtcttacgcgcagaagtttcaggg

SEQ ID NO: 20 - DNA, 60 bp, Homo sapiens
tgggtgagcgctatcaattatctttctagctatacctattatgcggatagcgtgaaaggc

SEQ ID NO: 21 - DNA, 60 bp, Homo sapiens
tggatgggcggtatccagccgatttttggcactgcgaattacgcgcagaagtttcagggc

SEQ ID NO: 22 - DNA, 60 bp, Homo sapiens
tggatgggcattatcgagccggatgatagctataccctttattctccgagctttcagggc

SEQ ID NO: 23 - DNA, 60 bp, Homo sapiens
tggatgggcggtatctctccgatttttggcactgcgaattacgcgcagaagtttcagggc

SEQ ID NO: 24 - DNA, 60 bp, Homo sapiens
tgggtgagcaatatctcttatggtggtagcaatacctattatgcggatagcgtgaaaggc

SEQ ID NO: 25 - DNA, 60 bp, Homo sapiens
tgggtgagcgttatctcttcttctggtagctatacctattatgcggatagcgtgaaaggc

SEQ ID NO: 26 - DNA, 60 bp, Homo sapiens
tggatgggcggtatcattccgcattttggcattgcgaattacgcgcagaagtttcagggc

SEQ ID NO: 27 - DNA, 60 bp, Homo sapiens
tggatgggcggtatcattccgtattttggcgctgcgaagtacgcgcagaagtttcagggc

SEQ ID NO: 28 - DNA, 60 bp, Homo sapiens
tggatgggcggtatcattccgatttttggcactgcgaattacgcgcagaagtttcagggc

SEQ ID NO: 29 - DNA, 60 bp, Homo sapiens
tggatgggcattatctatccgggtcatagctataccaattattctccgagctttcagggc

SEQ ID NO: 30 - DNA, 60 bp, Homo sapiens
tggatgggcggtatcattccgcattttggccatgcgtcttacgcgcagaagtttcagggc

SEQ ID NO: 31 - DNA, 60 bp, Homo sapiens
tgggtgagctatatctcttcttctggtagcattacctcttatgcggatagcgtgaaaggc

SEQ ID NO: 32 - DNA, 63 bp, Homo sapiens
tggctgggccgtatctattatcgtagcaagtggtataacgattatgcggtgagcgtgaaaagc

SEQ ID NO: 33 - DNA, 30 bp, Homo sapiens
ggatttaccttttcttcttatggtatgtct

SEQ ID NO: 34 - DNA, 30 bp, Homo sapiens
ggatttaccttttctaattattatatttct

SEQ ID NO: 35 - DNA, 30 bp, Homo sapiens
ggaggcacttttcgtaattatgctatttct

SEQ ID NO: 36 - DNA, 30 bp, Homo sapiens
ggatttaccttttcttcttatgctatgtct

SEQ ID NO: 37 - DNA, 30 bp, Homo sapiens
ggaggcactttttcttcttctgctatttct

SEQ ID NO: 38 - DNA, 30 bp, Homo sapiens
ggatattcctttactacttattggattggt

SEQ ID NO: 39 - DNA, 30 bp, Homo sapiens
ggaggcactttttcttctaatgctattcat

SEQ ID NO: 40 - DNA, 30 bp, Homo sapiens
ggatttaccttttctaattattggctttct

SEQ ID NO: 41 - DNA, 30 bp, Homo sapiens
ggatttaccttttcttcttattctatgaat

SEQ ID NO: 42 - DNA, 30 bp, Homo sapiens
ggaggcactttttcttcttatactatttct

SEQ ID NO: 43 - DNA, 30 bp, Homo sapiens
ggaggcactttttctgattatactatttct

SEQ ID NO: 44 - DNA, 29 bp, Homo sapiens
ggaggcactttttctaattatacttttct

SEQ ID NO: 45 - DNA, 30 bp, Homo sapiens
ggatattcctttacttcttattggattggt

SEQ ID NO: 46 - DNA, 29 bp, Homo sapiens
ggaggcacttttaattcttatacttttct

SEQ ID NO: 47 - DNA, 30 bp, Homo sapiens
ggatttaccttttctaattattggatttct

SEQ ID NO: 48 - DNA, 36 bp, Homo sapiens
ggagatagcgtgagcgataatactgctgcttggtct

SEQ ID NO: 49 - protein, 16 aa, Homo sapiens
EDSIFWRGWYWGAFDV

SEQ ID NO: 50 - protein, 12 aa, Homo sapiens
DHEVMHTMGFDV

SEQ ID NO: 51 - protein, 7 aa, Homo sapiens
YFMGYDH

SEQ ID NO: 52 - protein, 10 aa, Homo sapiens
MFLYYDFFDY

SEQ ID NO: 53 - protein, 10 aa, Homo sapiens
YGQQYFGYAY

SEQ ID NO: 54 - protein, 9 aa, Homo sapiens
LDAYTGFDH

SEQ ID NO: 55 - protein, 14 aa, Homo sapiens
WFYYLHPLFNGYFD

SEQ ID NO: 56 - protein, 8 aa, Homo sapiens
DYMYDFDV

SEQ ID NO: 57 - protein, 12 aa, Homo sapiens
GHIYDPGSNFDY

SEQ ID NO: 58 - protein, 15 aa, Homo sapiens
IYFNDTDSVLHDDAS

SEQ ID NO: 59 - protein, 6 aa, Homo sapiens
YGSFDN

SEQ ID NO: 60 - protein, 8 aa, Homo sapiens
SMYVYSPS

SEQ ID NO: 61 - protein, 11 aa, Homo sapiens
AINWDGSILDF

SEQ ID NO: 62 - protein, 9 aa, Homo sapiens
IDYYYGDDY

SEQ ID NO: 63 - protein, 12 aa, Homo sapiens
HPFQTYGYYADY

SEQ ID NO: 64 - protein, 11 aa, Homo sapiens
DGFYLGVPLDS

SEQ ID NO: 65 - protein, 20 aa, Homo sapiens
WVSGISGSSSLTSYADSVKG

SEQ ID NO: 66 - protein, 20 aa, Homo sapiens
WVSNISGSGSNTYYADSVKG

SEQ ID NO: 67 - protein, 20 aa, Homo sapiens
WMGGIIPEFGTASYAQKFQG

SEQ ID NO: 68 - protein, 20 aa, Homo sapiens
WVSAINYLSSYTYYADSVKG

SEQ ID NO: 69 - protein, 20 aa, Homo sapiens
WMGGIQPIFGTANYAQKFQG

SEQ ID NO: 70 - protein, 20 aa, Homo sapiens
WMGIIEPDDSYTLYSPSFQG

SEQ ID NO: 71 - protein, 20 aa, Homo sapiens
WMGGISPIFGTANYAQKFQG

SEQ ID NO: 72 - protein, 20 aa, Homo sapiens
WVSNISYGGSNTYYADSVKG

SEQ ID NO: 73 - protein, 20 aa, Homo sapiens
WVSVISSSGSYTYYADSVKG

SEQ ID NO: 74 - protein, 20 aa, Homo sapiens
WMGGIIPHFGIANYAQKFQG

SEQ ID NO: 75 - protein, 20 aa, Homo sapiens
WMGGIIPYFGAAKYAQKFQG

SEQ ID NO: 76 - protein, 20 aa, Homo sapiens
WMGGIIPIFGTANYAQKFQG

SEQ ID NO: 77 - protein, 20 aa, Homo sapiens
WMGIIYPGHSYTNYSPSFQG

SEQ ID NO: 78 - protein, 20 aa, Homo sapiens
WMGGIIPHFGHASYAQKFQG

SEQ ID NO: 79 - protein, 20 aa, Homo sapiens
WVSYISSSGSITSYADSVKG

SEQ ID NO: 80 - protein, 21 aa, Homo sapiens
WLGRIYYRSKWYNDYAVSVKS

SEQ ID NO: 81 - protein, 10 aa, Homo sapiens
GFTFSSYGMS

SEQ ID NO: 82 - protein, 10 aa, Homo sapiens
GFTFSNYYIS

SEQ ID NO: 83 - protein, 10 aa, Homo sapiens
GGTFRNYAIS

SEQ ID NO: 84 - protein, 10 aa, Homo sapiens
GFTFSSYAMS

SEQ ID NO: 85 - protein, 10 aa, Homo sapiens
GGTFSSSAIS

SEQ ID NO: 86 - protein, 10 aa, Homo sapiens
GYSFTTYWIG

SEQ ID NO: 87 - protein, 10 aa, Homo sapiens
GGTFSSNAIH

SEQ ID NO: 88 - protein, 10 aa, Homo sapiens
GFTFSNYWLS

SEQ ID NO: 89 - protein, 10 aa, Homo sapiens
GFTFSSYSMN

SEQ ID NO: 90 - protein, 10 aa, Homo sapiens
GGTFSSYTIS

SEQ ID NO: 91 - protein, 10 aa, Homo sapiens
GGTFSDYTIS

SEQ ID NO: 92 - protein, 10 aa, Homo sapiens
GGTFSNYTIS

SEQ ID NO: 93 - protein, 10 aa, Homo sapiens
GYSFTSYWIG

SEQ ID NO: 94 - protein, 10 aa, Homo sapiens
GGTFNSYTFS

SEQ ID NO: 95 - protein, 10 aa, Homo sapiens
GFTFSNYWIS

SEQ ID NO: 96 - protein, 12 aa, Homo sapiens
GDSVSDNTAAWS

SEQ ID NO: 97 - DNA, 27 bp, Homo sapiens
cagtcttatgattctattctttataat

SEQ ID NO: 98 - DNA, 29 bp, Homo sapiens
agtcttatgatattgatatgcaggctact

SEQ ID NO: 99 - DNA, 40 bp, Homo sapiens
ttattattgccagtcttttgatttttctgatgatcatgag

SEQ ID NO: 100 - DNA, 24 bp, Homo sapiens
cagcagtattctgatcttcctact

SEQ ID NO: 101 - DNA, 30 bp, Homo sapiens
gcttcttttgatcagcttggtaattctgtt

SEQ ID NO: 102 - DNA, 30 bp, Homo sapiens
cagtcttgggattctggttcttatcctaat

SEQ ID NO: 103 - DNA, 27 bp, Homo sapiens
tcttcttatgctcttaatgattctcat

SEQ ID NO: 104 - DNA, 30 bp, Homo sapiens
cagtcttatgattctacttctggttctcgt

SEQ ID NO: 105 - DNA, 30 bp, Homo sapiens
cagtcttatgattctcctcctgctattatt

SEQ ID NO: 106 - DNA, 30 bp, Homo sapiens
tcttatgattctactggtgattttattgtg

SEQ ID NO: 107 - DNA, 17 bp, Homo sapiens
tcttcttatgattttaa

SEQ ID NO: 108 - DNA, 30 bp, Homo sapiens
cagtcttttgatcctattcgttctaattat

SEQ ID NO: 109 - DNA, 24 bp, Homo sapiens
cagcagggtgataatcttcctatt

SEQ ID NO: 110 - DNA, 24 bp, Homo sapiens
atgcagtatggtgatgagcctaat

SEQ ID NO: 111 - DNA, 30 bp, Homo sapiens
ggtggttcttatgataattttggtggtgag

SEQ ID NO: 112 - DNA, 30 bp, Homo sapiens
tcttcttgggatatgatgaagtttacttat

SEQ ID NO: 113 - DNA, 33 bp, Homo sapiens
cttgtgatttatggtgattctgagcgtccctca

SEQ ID NO: 114 - DNA, 33 bp, Homo sapiens
cttgtgatttatgatgataataagcgtccctca

SEQ ID NO: 115 - DNA, 33 bp, Homo sapiens
cttatgatttatgatgttaataatcgtccctca

SEQ ID NO: 116 - DNA, 33 bp, Homo sapiens
ctattaatttataattcttcttctcgtgcaact

SEQ ID NO: 117 - DNA, 33 bp, Homo sapiens
cttctgatttatgctgatacttctcgtccctca

SEQ ID NO: 118 - DNA, 33 bp, Homo sapiens
cttgtgatttatgatgattctaatcgtccctca

SEQ ID NO: 119 - DNA, 36 bp, Homo sapiens
cttatgatttatggtggtgttaatcagcgtccctca

SEQ ID NO: 120 - DNA, 33 bp, Homo sapiens
cttgtgatttatgatacttctaatcgtccctca

SEQ ID NO: 121 - DNA, 33 bp, Homo sapiens
cttgtgatttataaggataataagcgtccctca

SEQ ID NO: 122 - DNA, 33 bp, Homo sapiens
atgatttattatgttaataatcgtccctcaggc

SEQ ID NO: 123 - DNA, 33 bp, Homo sapiens
cttatgatttatgaggtttcttctcgtccctca

SEQ ID NO: 124 - DNA, 33 bp, Homo sapiens
cttatgatttctggtgtttctcatcgtccctca

SEQ ID NO: 125 - DNA, 33 bp, Homo sapiens
ctattaatttatgatgcttcttctttgcaaagc

SEQ ID NO: 126 - DNA, 33 bp, Homo sapiens
ctattaatttatggtggttctaatcgtgcaact

SEQ ID NO: 127 - DNA, 33 bp, Homo sapiens
cttgtgatttatggtgattctcatcgtccctca

SEQ ID NO: 128 - DNA, 33 bp, Homo sapiens
cttgtgatttatgaggattctaatcgtccctca

SEQ ID NO: 129 - DNA, 33 bp, Homo sapiens
agcggcgatgctattcgttcttattatgctcat

SEQ ID NO: 130 - DNA, 33 bp, Homo sapiens
agcggcgatgctcttggtggtctgtatgtttat

SEQ ID NO: 131 - DNA, 42 bp, Homo sapiens
acgggtactagcagcgatgttggttcttattattatgtgtct

SEQ ID NO: 132 - DNA, 36 bp, Homo sapiens
agagcgagccagattgtttcttctgagtatctggct

SEQ ID NO: 133 - DNA, 39 bp, Homo sapiens
agcggcagcagcagcaacattggttcttattctgtgtat

SEQ ID NO: 134 - DNA, 33 bp, Homo sapiens
agcggcgattctcttggttcttattatgtttct

SEQ ID NO: 135 - DNA, 42 bp, Homo sapiens
acgggtactagcagcgatgttggtggttataattatgtgtct

SEQ ID NO: 136 - DNA, 33 bp, Homo sapiens
agcggcgataatcttggtaatcagtatgtttct

SEQ ID NO: 137 - DNA, 33 bp, Homo sapiens
agcggcgataatcttccttattattatgcttct

SEQ ID NO: 138 - DNA, 42 bp, Homo sapiens
acgggtactagcagcgatgttggttcttattatgtgtcttgg

SEQ ID NO: 139 - DNA, 42 bp, Homo sapiens
acgggtactagcagcgatgttggtttttatgatcgtgtgtct

SEQ ID NO: 140 - DNA, 42 bp, Homo sapiens
acggttactagcagcgatgttggtggttataattttgtgtct

SEQ ID NO: 141 - DNA, 33 bp, Homo sapiens
agagcgagcgagggtattctttcttggctgaat

SEQ ID NO: 142 - DNA, 36 bp, Homo sapiens
agagcgagccagtctgtttcttcttattttctggct

SEQ ID NO: 143 - DNA, 33 bp, Homo sapiens
agcggcgataatcttggtgattattatgcttct

SEQ ID NO: 144 - DNA, 33 bp, Homo sapiens
agcggcgataatcttcgttcttattatgctcat

SEQ ID NO: 145 - protein, 8 aa, Homo sapiens
SYDSILYN

SEQ ID NO: 146 - protein, 9 aa, Homo sapiens
SYDIDMQAT

SEQ ID NO: 147 - protein, 10 aa, Homo sapiens
QSFDFSDDHE

SEQ ID NO: 148 - protein, 8 aa, Homo sapiens
QQYSDLPT

SEQ ID NO: 149 - protein, 10 aa, Homo sapiens
ASFDQLGNSV

SEQ ID NO: 150 - protein, 10 aa, Homo sapiens
QSWDSGSYPN

SEQ ID NO: 151 - protein, 9 aa, Homo sapiens
SSYALNDSH

SEQ ID NO: 152 - protein, 10 aa, Homo sapiens
QSYDSTSGSR

SEQ ID NO: 153 - protein, 10 aa, Homo sapiens
QSYDSPPAII

SEQ ID NO: 154 - protein, 10 aa, Homo sapiens
SYDSTGDFIV

SEQ ID NO: 155 - protein, 8 aa, Homo sapiens
SSYDFNMF

SEQ ID NO: 156 - protein, 10 aa, Homo sapiens
QSFDPIRSNY

SEQ ID NO: 157 - protein, 8 aa, Homo sapiens
QQGDNLPI

SEQ ID NO: 158 - protein, 8 aa, Homo sapiens
MQYGDEPN

SEQ ID NO: 159 - protein, 10 aa, Homo sapiens
GGSYDNFGGE

SEQ ID NO: 160 - protein, 10 aa, Homo sapiens
SSWDMMKFTY

SEQ ID NO: 161 - protein, 12 aa, Homo sapiens
VLVIYGDSERPS

SEQ ID NO: 162 - protein, 12 aa, Homo sapiens
VLVIYDDNKRPS

SEQ ID NO: 163 - protein, 11 aa, Homo sapiens
LMIYDVNNRPS

SEQ ID NO: 164 - protein, 11 aa, Homo sapiens
LLIYNSSSRAT

SEQ ID NO: 165 - protein, 11 aa, Homo sapiens
LLIYADTSRPS

SEQ ID NO: 166 - protein, 11 aa, Homo sapiens
LVIYDDSNRPS

SEQ ID NO: 167 - protein, 12 aa, Homo sapiens
LMIYGGVNQRPS

SEQ ID NO: 168 - protein, 11 aa, Homo sapiens
LVIYDTSNRPS

SEQ ID NO: 169 - protein, 11 aa, Homo sapiens
LVIYKDNKRPS

SEQ ID NO: 170 - protein, 12 aa, Homo sapiens
MIYYVNNRPSGV

SEQ ID NO: 171 - protein, 11 aa, Homo sapiens
LMIYEVSSRPS

SEQ ID NO: 172 - protein, 11 aa, Homo sapiens
LMISGVSHRPS

SEQ ID NO: 173 - protein, 11 aa, Homo sapiens
LLIYDASSLQS

SEQ ID NO: 174 - protein, 11 aa, Homo sapiens
LLIYGGSNRAT

SEQ ID NO: 175 - protein, 11 aa, Homo sapiens
LVIYGDSHRPS

SEQ ID NO: 176 - protein, 11 aa, Homo sapiens
LVIYEDSNRPS

SEQ ID NO: 177 - protein, 11 aa, Homo sapiens
SGDAIRSYYAH

SEQ ID NO: 178 - protein, 11 aa, Homo sapiens
SGDALGGLYVY

SEQ ID NO: 179 - protein, 14 aa, Homo sapiens
TGTSSDVGSYYYVS

SEQ ID NO: 180 - protein, 12 aa, Homo sapiens
RASQIVSSEYLA

SEQ ID NO: 181 - protein, 13 aa, Homo sapiens
SGSSSNIGSYSVY

SEQ ID NO: 182 - protein, 11 aa, Homo sapiens
SGDSLGSYYVS

SEQ ID NO: 183 - protein, 14 aa, Homo sapiens
TGTSSDVGGYNYVS

SEQ ID NO: 184 - protein, 11 aa, Homo sapiens
SGDNLGNQYVS

SEQ ID NO: 185 - protein, 11 aa, Homo sapiens
SGDNLPYYYAS

SEQ ID NO: 186 - protein, 14 aa, Homo sapiens
TGTSSDVGSYYVSW

SEQ ID NO: 187 - protein, 14 aa, Homo sapiens
TGTSSDVGFYDRVS

SEQ ID NO: 188 - protein, 14 aa, Homo sapiens
TVTSSDVGGYNFVS

SEQ ID NO: 189 - protein, 11 aa, Homo sapiens
RASEGILSWLN

SEQ ID NO: 190 - protein, 12 aa, Homo sapiens
RASQSVSSYFLA

SEQ ID NO: 191 - protein, 11 aa, Homo sapiens
SGDNLGDYYAS

SEQ ID NO: 192 - protein, 11 aa, Homo sapiens
SGDNLRSYYAH

SEQ ID NO: 193 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDTMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 194 - DNA, 66 bp, Homo sapiens
tgggtgagccttattgaggctaaggctggtaattatgctactgattatgctgcttctgttaagggt

SEQ ID NO: 195 - DNA, 66 bp, Homo sapiens
tgggtgagctttattgagggtaagactactggttatgctactgattatgctgcttctgttaagggt

SEQ ID NO: 196 - DNA, 60 bp, Homo sapiens; identical to SEQ ID NO: 17

SEQ ID NO: 197 - DNA, 60 bp, Homo sapiens; identical to SEQ ID NO: 17

SEQ ID NO: 198 - DNA, 60 bp, Homo sapiens; identical to SEQ ID NO: 17

SEQ ID NO: 199 - DNA, 60 bp, Homo sapiens; identical to SEQ ID NO: 17

SEQ ID NO: 200 - DNA, 66 bp, Homo sapiens; identical to SEQ ID NO: 194

SEQ ID NO: 201 - DNA, 54 bp, Homo sapiens
tgggtgagcgttatttctggtggtcctactttttatgctgattctgttaagggt

SEQ ID NO: 202 - DNA, 57 bp, Homo sapiens
tgggtgagcgttatttttaatgttggtggtacttattatgctgattctgttaagggt

SEQ ID NO: 203 - DNA, 60 bp, Homo sapiens
tgggtgagcgctatttggggtaagggttctgttaagttttatgctgattctgttaagggt

SEQ ID NO: 204 - DNA, 60 bp, Homo sapiens; identical to SEQ ID NO: 25

SEQ ID NO: 205 - DNA, 60 bp, Homo sapiens; identical to SEQ ID NO: 25

SEQ ID NO: 206 - DNA, 60 bp, Homo sapiens
tgggtgagcgttatttctggtggtcctactttttatgctgattctgttaagggtcgtttt

SEQ ID NO: 207 - DNA, 60 bp, Homo sapiens
tgggtgagcgttatttttaatgttggtggtacttattatgctgattctgttaagggtcgt

SEQ ID NO: 208 - DNA, 60 bp, Homo sapiens; identical to SEQ ID NO: 203

SEQ ID NO: 209 - DNA, 60 bp, Homo sapiens; identical to SEQ ID NO: 206

SEQ ID NO: 210 - DNA, 60 bp, Homo sapiens; identical to SEQ ID NO: 207

SEQ ID NO: 211 - DNA, 60 bp, Homo sapiens; identical to SEQ ID NO: 203

SEQ ID NO: 212 - DNA, 60 bp, Homo sapiens
tggatgggcattattaagccttctctttcttatactgtttattctccttcttttcagggt

SEQ ID NO: 213 - DNA, 60 bp, Homo sapiens
tggatgggcattattaagcctgctatgtcttatactgtttattctccttcttttcagggt

SEQ ID NO: 214 - DNA, 60 bp, Homo sapiens; identical to SEQ ID NO: 29

SEQ ID NO: 215 - DNA, 60 bp, Homo sapiens; identical to SEQ ID NO: 29

SEQ ID NO: 216 - DNA, 60 bp, Homo sapiens; identical to SEQ ID NO: 212

SEQ ID NO: 217 - DNA, 60 bp, Homo sapiens; identical to SEQ ID NO: 213

SEQ ID NO: 218 - DNA, 60 bp, Homo sapiens; identical to SEQ ID NO: 17

SEQ ID NO: 219 - DNA, 60 bp, Homo sapiens; identical to SEQ ID NO: 20

SEQ ID NO: 220 - DNA, 60 bp, Homo sapiens; identical to SEQ ID NO: 20

SEQ ID NO: 221 - DNA, 60 bp, Homo sapiens
tgggtgagctatatttcttttactggtcgtaatattaattatgctgattctgttaagggt

SEQ ID NO: 222 - DNA, 60 bp, Homo sapiens
tgggtgagctatatttcttggaatggtaagtttatttattatgctgattctgttaagggt

SEQ ID NO: 223 - DNA, 60 bp, Homo sapiens
tgggtgagctatatttcttgggctggtgatcttactaattatgctgattctgttaagggt

SEQ ID NO: 224 - DNA, 60 bp, Homo sapiens; identical to SEQ ID NO: 30

SEQ ID NO: 225 - DNA, 60 bp, Homo sapiens; identical to SEQ ID NO: 30

SEQ ID NO: 226 - DNA, 63 bp, Homo sapiens
tggctgggccgtacttattatcgttctgcttggcatcgtcattatgctgattctgttaagtct

SEQ ID NO: 227 - DNA, 63 bp, Homo sapiens
tggctgggccgtacttattatcgtggtaattggtatcgtcattatgctgcttctgttaagtct

SEQ ID NO: 228 - protein, 12 aa, Homo sapiens
WVSLIEAKAGNY

SEQ ID NO: 229 - protein, 22 aa, Homo sapiens
WVSFIEGKTTGYATDYAASVKG

SEQ ID NO: 230 - protein, 22 aa, Homo sapiens
EWVSGISGSSSLTSYADSVKGR

SEQ ID NO: 231 - protein, 21 aa, Homo sapiens
EWVSGISGSSSLTSYADSVKG

SEQ ID NO: 232 - protein, 21 aa, Homo sapiens
EWVSGISGSSSLTSYADSVKG

SEQ ID NO: 233 - protein, 20 aa, Homo sapiens
WVSGISGSSSLTSYADSVKG

SEQ ID NO: 234 - protein, 22 aa, Homo sapiens
WVSLIEAKAGNYATDYAASVKG

SEQ ID NO: 235 - protein, 18 aa, Homo sapiens
WVSVISGGPTFYADSVKG

SEQ ID NO: 236 - protein, 19 aa, Homo sapiens
WVSVIFNVGGTYYADSVKG

SEQ ID NO: 237 - protein, 20 aa, Homo sapiens
WVSAIWGKGSVKFYADSVKG

SEQ ID NO: 238 - protein, 20 aa, Homo sapiens
WVSVISSSGSYTYYADSVKG

SEQ ID NO: 239 - protein, 20 aa, Homo sapiens
WVSVISSSGSYTYYADSVKG

SEQ ID NO: 240 - protein, 20 aa, Homo sapiens
WVSVISGGPTFYADSVKGRF

SEQ ID NO: 241 - protein, 20 aa, Homo sapiens
WVSVISGGPTFYADSVKGRF

SEQ ID NO: 242 - protein, 20 aa, Homo sapiens
WVSAIWGKGSVKFYADSVKG

SEQ ID NO: 243 - protein, 20 aa, Homo sapiens
WVSVISGGPTFYADSVKGRF

SEQ ID NO: 244 - protein, 17 aa, Homo sapiens
WVSVIFNVGGTYYADSV

SEQ ID NO: 245 - protein, 20 aa, Homo sapiens
WVSAIWGKGSVKFYADSVKG

SEQ ID NO: 246 - protein, 21 aa, Homo sapiens
WMGIIKPSLSYTVYSPSFQGQ

SEQ ID NO: 247 - protein, 20 aa, Homo sapiens
WMGIIKPAMSYTVYSPSFQG

SEQ ID NO: 248 - protein, 20 aa, Homo sapiens
WMGIIYPGHSYTNYSPSFQG

SEQ ID NO: 249 - protein, 20 aa, Homo sapiens
WMGIIYPGHSYTNYSPSFQG

SEQ ID NO: 250 - protein, 20 aa, Homo sapiens
WMGIIKPSLSYTVYSPSFQG

SEQ ID NO: 251 - protein, 20 aa, Homo sapiens
WMGIIKPAMSYTVYSPSFQG

SEQ ID NO: 252 - protein, 20 aa, Homo sapiens
WVSGISGSSSLTSYADSVKG

SEQ ID NO: 253 - protein, 20 aa, Homo sapiens
WVSAINYLSSYTYYADSVKG

SEQ ID NO: 254 - protein, 20 aa, Homo sapiens
WVSAINYLSSYTYYADSVKG

SEQ ID NO: 255 - protein, 20 aa, Homo sapiens
WVSYISFTGRNINYADSVKG

SEQ ID NO: 256 - protein, 20 aa, Homo sapiens
WVSYISWNGKFIYYADSVKG

SEQ ID NO: 257 - protein, 20 aa, Homo sapiens
WVSYISWAGDLTNYADSVKG

SEQ ID NO: 258 - protein, 20 aa, Homo sapiens
WMGGIIPHFGHASYAQKFQG

SEQ ID NO: 259 - protein, 21 aa, Homo sapiens
EWMGGIIPHFGHASYAQKFQG

SEQ ID NO: 260 - protein, 21 aa, Homo sapiens
WLGRTYYRSAWHRHYADSVKS

SEQ ID NO: 261 - protein, 21 aa, Homo sapiens
WLGRTYYRGNWYRHYAASVKS

SEQ ID NO: 262 - DNA, 27 bp, Homo sapiens
cagtcttatgattctattctttataat

SEQ ID NO: 263 - DNA, 27 bp, Homo sapiens
cagtcttatgattctattctttataat

SEQ ID NO: 264 - DNA, 30 bp, Homo sapiens
tcttcttttgatcaggtttcttatggtgat

SEQ ID NO: 265 - DNA, 30 bp, Homo sapiens
tcttcttttgatgattcttcttctggttct

SEQ ID NO: 266 - DNA, 30 bp, Homo sapiens
tcttcttatgatgaggatgatgttggttct

SEQ ID NO: 267 - DNA, 27 bp, Homo sapiens
tcttcttatgatggttcttatggtcat

SEQ ID NO: 268 - DNA, 30 bp, Homo sapiens; identical to SEQ ID NO: 265

SEQ ID NO: 269 - DNA, 30 bp, Homo sapiens; identical to SEQ ID NO: 105

SEQ ID NO: 270 - DNA, 30 bp, Homo sapiens; identical to SEQ ID NO: 105

SEQ ID NO: 271 - DNA, 30 bp, Homo sapiens; identical to SEQ ID NO: 105

SEQ ID NO: 272 - DNA, 30 bp, Homo sapiens
cagtcttatgatgagattcctttttttcct

SEQ ID NO: 273 - DNA, 27 bp, Homo sapiens
cagtcttatactgagctttttcatcct

SEQ ID NO: 274 - DNA, 30 bp, Homo sapiens; identical to SEQ ID NO: 272

SEQ ID NO: 275 - DNA, 30 bp, Homo sapiens; identical to SEQ ID NO: 272

SEQ ID NO: 276 - DNA, 30 bp, Homo sapiens; identical to SEQ ID NO: 272

SEQ ID NO: 277 - DNA, 27 bp, Homo sapiens
cagtcttatactgagctttttcatcct

SEQ ID NO: 278 - DNA, 27 bp, Homo sapiens
cagtcttatactgagctttttcatcct

SEQ ID NO: 279 - DNA, 27 bp, Homo sapiens
cagtcttatactgagctttttcatcct

SEQ ID NO: 280 - DNA, 24 bp, Homo sapiens
cagcagggtgataatcttcctatt

SEQ ID NO: 281 - DNA, 24 bp, Homo sapiens
cagcagggtgataatcttcctatt

SEQ ID NO: 282 - DNA, 24 bp, Homo sapiens
cagcagtctattgatcttcctttt

SEQ ID NO: 283 - DNA, 24 bp, Homo sapiens
cagcagggtggtactattcctttt

SEQ ID NO: 284 - DNA, 24 bp, Homo sapiens
cagcagtctattgatcttcctttt

SEQ ID NO: 285 - DNA, 24 bp, Homo sapiens
cagcagtctattgatcttcctttt

SEQ ID NO: 286 - DNA, 24 bp, Homo sapiens
cagtcttatgatgagcctggtgat

SEQ ID NO: 287 - DNA, 24 bp, Homo sapiens
cagcagtatattactattcctcct

SEQ ID NO: 288 - DNA, 24 bp, Homo sapiens
cagcagtatatttctattcctcct

SEQ ID NO: 289 - DNA, 30 bp, Homo sapiens; identical to SEQ ID NO: 104

SEQ ID NO: 290 - DNA, 30 bp, Homo sapiens; identical to SEQ ID NO: 104

SEQ ID NO: 291 - DNA, 30 bp, Homo sapiens; identical to SEQ ID NO: 104

SEQ ID NO: 292 - DNA, 27 bp, Homo sapiens
cagcagtattttactgatcctgagttt

SEQ ID NO: 293 - DNA, 24 bp, Homo sapiens
cagcagtattttcatgagcctctt

SEQ ID NO: 294 - DNA, 30 bp, Homo sapiens; identical to SEQ ID NO: 112

SEQ ID NO: 295 - DNA, 30 bp, Homo sapiens; identical to SEQ ID NO: 112

SEQ ID NO: 296 - protein, 9 aa, Homo sapiens
QSYDSILYN

SEQ ID NO: 297 - protein, 9 aa, Homo sapiens
QSYDSILYN

SEQ ID NO: 298 - protein, 10 aa, Homo sapiens
SSFDQVSYGD

SEQ ID NO: 299 - protein, 10 aa, Homo sapiens
SSFDDSSSGS

SEQ ID NO: 300 - protein, 10 aa, Homo sapiens
SSYDEDDVGS

SEQ ID NO: 301 - protein, 9 aa, Homo sapiens
SSYDGSYGH

SEQ ID NO: 302 - protein, 10 aa, Homo sapiens
SSFDDSSSGS

SEQ ID NO: 303 - protein, 10 aa, Homo sapiens
QSYDSPPAII

SEQ ID NO: 304 - protein, 10 aa, Homo sapiens
QSYDSPPAII

SEQ ID NO: 305 - protein, 10 aa, Homo sapiens
QSYDSPPAII

SEQ ID NO: 306 - protein, 10 aa, Homo sapiens
QSYDEIPFFP

SEQ ID NO: 307 - protein, 9 aa, Homo sapiens
QSYTELFHP

SEQ ID NO: 308 - protein, 10 aa, Homo sapiens
QSYDEIPFFP

SEQ ID NO: 309 - protein, 10 aa, Homo sapiens
QSYDEIPFFP

SEQ ID NO: 310 - protein, 10 aa, Homo sapiens
QSYDEIPFFP

SEQ ID NO: 311 - protein, 9 aa, Homo sapiens
QSYTELFHP

SEQ ID NO: 312 - protein, 9 aa, Homo sapiens
QSYTELFHP

SEQ ID NO: 313 - protein, 9 aa, Homo sapiens
QSYTELFHP

SEQ ID NO: 314 - protein, 8 aa, Homo sapiens
QQGDNLPI

SEQ ID NO: 315 - protein, 8 aa, Homo sapiens
QQGDNLPI

SEQ ID NO: 316 - protein, 8 aa, Homo sapiens
QQSIDLPF

SEQ ID NO: 317 - protein, 8 aa, Homo sapiens
QQGGTIPF

SEQ ID NO: 318 - protein, 8 aa, Homo sapiens
QQSIDLPF

SEQ ID NO: 319 - protein, 8 aa, Homo sapiens
QQSIDLPF

SEQ ID NO: 320 - protein, 8 aa, Homo sapiens
QSYDEPGD

SEQ ID NO: 321 - protein, 8 aa, Homo sapiens
QQYITIPP

SEQ ID NO: 322 - protein, 8 aa, Homo sapiens
QQYISIPP

SEQ ID NO: 323 - protein, 10 aa, Homo sapiens
QSYDSTSGSR

SEQ ID NO: 324 - protein, 10 aa, Homo sapiens
QSYDSTSGSR

SEQ ID NO: 325 - protein, 10 aa, Homo sapiens
QSYDSTSGSR

SEQ ID NO: 326 - protein, 9 aa, Homo sapiens
QQYFTDPEF

SEQ ID NO: 327 - protein, 8 aa, Homo sapiens
QQYFHEPL

SEQ ID NO: 328 - protein, 10 aa, Homo sapiens
SSWDMMKFTY

SEQ ID NO: 329 - protein, 10 aa, Homo sapiens
SSWDMMKFTY

(19) United States
(12) Patent Application Publication
(10) Pub. No.: US 2011/0110944 A1
(43) Pub. Date: May 12, 2011

(54) ANTI-ALK1 ANTIBODIES AND USES THEREOF

(75) Inventors: Stefan Steidl, Munich (DE); Corinne Petit-Frère, Munich (DE); Iring Heisler, Wuppertal (DE); Dmitry Zubov, Remscheid (DE); Verena Voehringer, Wuppertal (DE); Karina Schuck, Mannheim (DE)

(73) Assignee: MORPHOSYS AG, Martinsried/Planegg (unknown)

(21) Appl. No.: 12/988,521

(22) PCT Filed: Apr. 24, 2009
(86) PCT No.: PCT/EP2009/054936

§ 371 Date: Jan. 13, 2011

(30) Foreign Application Priority Data
Apr. 25, 2008 (EP) 08 00 8019.5

(51) Int. Cl. A61K 39/395; C07K 16/28; C07H 21/04; C12N 15/63; C12N 5/10; C12N 1/21; A61P 35/00; A61P 27/00; C40B 30/04; C40B 40/10; C12N 5/00

(52) U.S. Cl. 424/133.1; 530/387.9; 530/387.3; 536/23.53; 435/320.1; 435/325; 435/252.3; 424/139.1; 506/9; 506/18; 435/375

(57) ABSTRACT

The present invention provides recombinant antigen-binding regions and antibodies and functional fragments containing such antigen-binding regions that are specific for AIk1, which plays an integral role in various disorders or conditions, such as cancer and macular degeneration. These antibodies, accordingly, can be used to treat these and other disorders and conditions. Antibodies of the invention also can be used in the diagnostics field, as well as for further investigating the role of AIk1 in the progression of disorders associated with pathogenic angiogenesis. The invention also provides nucleic acid sequences encoding the foregoing antibodies, vectors containing the same, pharmaceutical compositions and kits with instructions for use.

The present invention provides recombinant antigen-binding regions and antibodies and functional fragments thereof containing such antigen-binding regions that are specific for Alk1, which plays an integral role in various disorders or conditions, such as cancer and macular degeneration. These antibodies, accordingly, can be used to treat these and other disorders and conditions. Antibodies, or functional fragments thereof, of the invention also can be used in the diagnostics field, as well as for further investigating the role of Alk1 in the progression of disorders associated with pathogenic angiogenesis. The invention also provides nucleic acid sequences encoding the foregoing antibodies, vectors containing the same, pharmaceutical compositions and kits with instructions for use.

### BACKGROUND OF THE INVENTION

Members of the TGFβ family mediate their biological activity by binding to a heterodimeric receptor complex of type I and type II serine/threonine kinase receptors TβRI and TβRII. The overall structure of type I and type II receptors are similar: they are composed of small cysteine-rich extra cellular parts, single transmembrane regions and intracellular parts that contain serin/threonine kinase domains. TGFβ ligands have a high affinity for the TβRII and upon binding to this receptor, a specific TβRI is recruited. Once this receptor complex is formed, the GS domain of the TβRI is phosphorylated by the TβRII, resulting in a conformational change.

The type I receptor is also known as Activin receptor like kinase (ALK). In most cells TGFβ signals through ALK5 but in endothelial cells TGFβ signaling occurs through ALK1 and ALK5 (Goumans et al, 2002; 2003). Activated ALK5 leads to phosphorylation of smad2/3 while ALK1 activation results in phosphorylation of smad1/5. Type III receptors (also called accessory TGFβ receptors) such as endoglin and b-glycan have a more indirect role in TGFβ signaling.

Endoglin does not bind to TGFβ on its own but interacts with the TβRII. Its role in the endothelial cell receptor complex is not completely understood but it has been suggested that endoglin stimulates TGFβ/ALK1 signaling either by recruiting ALK1 to the receptor complex or by stimulating ALK1 kinase activity. (Lebrin et al EMBO J 2004; Koleva et al, 2006).

Mutations in endoglin and in ALK1 are linked to the autosomal dominant disorder called hereditary hemorrhagic telangectasia (HHT1 and HHT2 respectively). The characteristics of the disease are telangiectases consisting of focal dilatations of post capillary venules and arterial venous malformations (Fernandez-L, et al 2006).

The physiological ligand of ALK1 is still debated in literature and TGFβ as well as BMP-9 (and BMP-10) have been proposed (Lebrin et al, 2005; Brown et al, 2005; David et al 2007b; Scharpfenecker et al, 2007).

Brown et al (2005) have determined the crystal structure of BMP9 and identified ALK1 as its potential receptor. In addition, David et al (2007b) showed that in microvascular endothelial cells, upon binding of BMP9 to ALK1, the smad1/2/8 pathway is stimulated. David et al.(2007b) postulate that BMP9 inhibits endothelial cell migration and growth.

There is a controversial discussion in literature between the group of P. ten Dijke and F. Bailly regarding the endothelium-activating function of Alk1 (e.g. Goumans 2002/2003, Lebrin et al., 2004/2005, Scharpfenecker 2007 vs. Mallet et al 2006, David et al 2007a, Lamouille at al 2002). Evidence, mainly from the ten Dijke group, suggests that both signaling pathways have opposite effects on endothelial cells: ALK5 inhibits endothelial cell migration and proliferation while ALK1 stimulates both processes. In addition ALK1 is negative regulator of the TGFβ/ALK5 signaling pathway and ALK5 is essential for efficient ALK1 activation (Goumans 2003).

### SUMMARY OF THE INVENTION

It is an object of the invention to provide human and humanized antibodies against Alk1.

It is another object of the invention to provide antibodies that are safe for human administration.

It is also an object of the present invention to provide methods for treating disease or and/or conditions associated with Alk1 by using one or more antibodies against Alk1, such as the antibodies described in the present invention. These and other objects of the invention are more fully described herein.

In one aspect, the invention provides an isolated antibody or functional antibody fragment that contains an antigen-binding region that is specific for an epitope of Alk1.

In another aspect, the invention provides an isolated human or humanized antibody or functional fragment thereof comprising an antigen-binding region that is specific for Alk1 (SEQ ID NO: 193), wherein said antibody or functional fragment thereof possesses one or more of the following properties: a) is able to inhibit proliferation of endothelial cells, b) produce specific staining of tumor blood vessels in IHC, c) inhibit heterodimerization of Alk1 receptor d) inhibit BMP9 stimulated induction of SMAD7 expression in endothelial cells, e) inhibit BMP9 stimulated SMAD1/5/8 phosphorylation f) possesses a specific affinity (Kd) at or below 1 nmol as determined by BIACORE or SET, g) inhibit angiogenesis, h) inhibit tumor growth, i) inhibit endothelial cell migration, j) inhibit smooth muscle cell proliferation, k) inhibit endothelial cell tube formation, l) induces ADCC in vivo. Such an antibody or functional fragment thereof may contain an antigen-binding region that contains an H-CDR3 region depicted in SEQ ID NOS: 1-16, 49-64; the antigen-binding region may further include an H-CDR2 region depicted in SEQ ID NOS: 17-32, 56-80, 194-227, 228-261; and the antigen-binding region also may contain an H-CDR1 region depicted in SEQ ID NOS: 33-48, 81-96. Such a Alk1-specific antibody of the invention may contain an antigen-binding region that contains an L-CDR3 region depicted in SEQ ID NOS: 97-112, 145-160, 262-295, 296-329; the antigen-binding region may further include an L-CDR1 region depicted in SEQ ID NOS: 129-144, 177-192; and the antigen-binding region also may contain an L-CDR2 region depicted in SEQ ID NOS: 113-128, 161-176.

Antibodies (and functional fragments thereof) of the invention may contain an antigen-binding region that is specific for an epitope of Alk1, which epitope contains one or more amino acid residues of amino acid residues 20 to 118 of Alk1, as depicted by SEQ ID NO: 193 For certain antibodies, the epitope may be linear, whereas for others, it may be conformational (i.e., discontinuous).

An antibody or functional fragment thereof having one or more of these properties may contain an antigen-binding region that contains an H-CDR3 region depicted in ID's NO: 1-16, 49-64, 194-227, 228-261; the antigen-binding region may further include an H-CDR2 region depicted in SEQ ID NOS: 17-32, 56-80; and the antigen-binding region also may contain an H-CDR1 region depicted in SEQ ID NOS: 33-48, 81-96. Such a Alk1-specific antibody of the invention may contain an antigen-binding region that contains an L-CDR3 region depicted in SEQ ID NOS: 97-112, 145-160, 262-295, 296-329; the antigen-binding region may further include an L-CDR1 region depicted in ID NOS: 129-144, 177-192; and the antigen-binding region also may contain an L-CDR2 region depicted in SEQ ID NOS: 113-128, 161-176.

Peptide variants of the sequences disclosed herein are also embraced by the present invention. Accordingly, the invention includes anti-Alk1 antibodies having a heavy chain amino acid sequence with: at least 60 percent sequence identity in the CDR regions with the CDR regions depicted in ID's NOS: 49-64, 56-80, 81-96, 228-261; at least 70 percent sequence identity in the CDR regions with the CDR regions depicted in ID's NOS: 49-64, 56-80, 81-96, 228-261; at least 80 percent sequence identity in the CDR regions with the CDR regions depicted in ID's NOS: 49-64, 56-80, 81-96, 228-261; at least 90 percent sequence identity in the CDR regions with the CDR regions depicted in ID's NOS: 49-64, 56-80, 81-96, 228-261; and/or at least 95 percent sequence homology in the CDR regions with the CDR regions depicted in SEQ ID NOS: 49-64, 56-80, 81-96, 228-261. Further included are anti-Alk1 antibodies having a light chain amino acid sequence with: at least 60 percent sequence identity in the CDR regions with the CDR regions depicted in SEQ ID NOS: 145-160, 177-192, 161-176, 296-329; at least 70 percent sequence identity in the CDR regions with the CDR regions depicted in SEQ ID NOS: 145-160, 177-192, 161-176, 296-329; at least 80 percent sequence identity in the CDR regions with the CDR regions depicted in SEQ ID NOS: 145-160, 177-192, 161-176, 296-329; at least 90 percent sequence identity in the CDR regions with the CDR regions depicted in SEQ ID NOS: 145-160, 177-192, 161-176, 296-329; and/or at least 95 percent sequence homology in the CDR regions with the CDR regions depicted in SEQ ID NOS: 145-160, 177-192, 161-176, 296-329.

An antibody of the invention may be an IgG (e.g., IgG₁), while an antibody fragment may be a Fab or scFv, for example. An inventive antibody fragment, accordingly, may be, or may contain, an antigen-binding region that behaves in one or more ways as described herein.

The invention also is related to isolated nucleic acid sequences, each of which can encode an antigen-binding region of a human antibody or functional fragment thereof that is specific for an epitope of Alk1. Such a nucleic acid sequence may encode a variable heavy chain of an antibody and include a sequence selected from the group consisting of SEQ ID NOS: 1-48, 194-227, or a nucleic acid sequence that hybridizes under high stringency conditions to the complementary strand of SEQ ID NOS: 1-48, 194-227. The nucleic acid might encode a variable light chain of an isolated antibody or functional fragment thereof, and may contain a sequence selected from the group consisting of SEQ ID NOS: 97-144, 262-295 or a nucleic acid sequence that hybridizes under high stringency conditions to the complementary strand of SEQ ID NOS: 97-144, 262-295.

Nucleic acids of the invention are suitable for recombinant production. Thus, the invention also relates to vectors and host cells containing a nucleic acid sequence of the invention.

Compositions of the invention may be used for therapeutic or prophylactic applications. The invention, therefore, includes a pharmaceutical composition containing an inventive antibody (or functional antibody fragment) and a pharmaceutically acceptable carrier or excipient therefor. In a related aspect, the invention provides a method for treating a disorder or condition associated with the undesired presence of Alk1 or Alk1 expressing cells. Such method contains the steps of administering to a subject in need thereof an effective amount of the pharmaceutical composition that contains an inventive antibody as described or contemplated herein.

The invention also relates to isolated epitopes of Alk1, either in linear or conformational form, and their use for the isolation of an antibody or functional fragment thereof, which antibody or antibody fragment comprises an antigen-binding region that is specific for said epitope. In this regard, a linear epitope may contain amino acid residues 20-118, while a conformational epitope may contain one or more amino acid residues selected from the group consisting of amino acids 20-118 of Alk1 (SEQ ID NO 193). An epitope of Alk1 can be used, for example, for the isolation of antibodies or functional fragments thereof (each of which antibodies or antibody fragments comprises an antigen-binding region that is specific for such epitope), comprising the steps of contacting said epitope of Alk1 with an antibody library and isolating the antibody(ies) or functional fragment(s) thereof.

In another embodiment, the invention provides an isolated epitope of Alk1, which consists essentially of an amino acid sequence selected from the group consisting of amino acids 20-118 of Alk1 (SEQ ID NO 193). In an alternative embodiment, the invention provides an isolated epitope of Alk1, which comprises an amino acid sequence selected from the group consisting of amino acids 20-118 of Alk1 (SEQ ID NO 193). As used herein, such an epitope “consists essentially of” one of the immediately preceding amino acid sequences plus additional features, provided that the additional features do not materially affect the basic and novel characteristics of the epitope.

In yet another embodiment, the invention provides an isolated epitope of Alk1 that consists of an amino acid sequence selected from the group consisting of amino acids 20-118 of Alk1 (SEQ ID NO 193).

The invention also provides a kit containing (i) an isolated epitope of Alk1 comprising one or more amino acid stretches taken from the list of amino acids 20-118 of Alk1 (SEQ ID NO 193); (ii) an antibody library; and (iii) instructions for using the antibody library to isolate one or more members of such library that binds specifically to such epitope.

### BRIEF DESCRIPTION OF THE FIGURES

FIG. 1a provides nucleic acid sequences of various parental antibody variable heavy regions.

FIG. 1b provides amino acid sequences of various parental antibody variable heavy regions. CDR regions HCDR1, HCDR2 and HCDR3 are designated from N- to C-terminus in boldface.

FIG. 2 provides nucleic acid sequences of various parental antibody variable light regions.

FIG. 3 provides amino acid sequences of various parental antibody variable light regions. CDR regions LCDR1, LCDR2 and LCDR3 are designated from N- to C-terminus in boldface.

FIG. 4a provides nucleic acid sequences of various maturated antibody variable heavy regions.

FIG. 4b provides amino acid sequences of various maturated antibody variable heavy regions. CDR regions HCDR2 are designated from N- to C-terminus in boldface.

FIG. 4c provides nucleic acid sequences of various maturated antibody variable light regions.

FIG. 4d provides amino acid sequences of various maturated antibody variable light regions. CDR regions LCDR3 are designated from N- to C-terminus in boldface.

FIG. 5 provides the amino acid sequence of Alk1 (SEQ ID NO 193).

FIG. 6 provides the exemplary immunohistochemistry analysis of breast carcinoma sections using parental and maturated Fab-dHLX antibodies. The MOR numbers of the antibodies used are indicated below the individual panels. “CD31” shows results with an antibody specific for endothelial cells. “Control” refers to control staining without antibody.

FIG. 7 provides exemplary Biacore kₒₙ and k_{off} curve fits for Fab MOR06326.

FIG. 8 Provides the data on the ability of selected antibodies to inhibit VEGF-C induced proliferation of MVEC cells.

FIG. 9 Provides the data on the ability of selected antibodies to inhibit VEGF-C induced proliferation of HMVEC cells.

FIG. 10 Provides the data on the ability of selected antibodies to inhibit VEGF-A induced proliferation of HMVEC cells.

FIG. 11 Provides the data on the ability of selected antibodies to inhibit BMP9 induced induction of SMAD7 expression in HMEC-1 cells (FIG. 11a) and HDMEC-A cells (FIG. 11b)

### DETAILED DESCRIPTION OF THE INVENTION

The present invention is based on the discovery of novel antibodies that are specific to or have a high affinity for Alk1 and can deliver a therapeutic benefit to a subject. The antibodies of the invention, which may be human or humanized, can be used in many contexts, which are more fully described herein.

A “human” antibody or functional human antibody fragment is hereby defined as one that is not chimeric (e.g., not “humanized”) and not from (either in whole or in part) a non-human species. A human antibody or functional antibody fragment can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained therefrom. Another example of a human antibody or functional antibody fragment, is one that is encoded by a nucleic acid isolated from a library of antibody sequences of human origin (i.e., such library being based on antibodies taken from a human natural source).

A “humanized antibody” or functional humanized antibody fragment is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a non-human origin, while one or more frameworks of the variable domain are of human origin and the constant domain (if any) is of human origin.

As used herein, an antibody “binds specifically to,” is “specific to/for” or “specifically recognizes” an antigen (here, Alk1) if such antibody is able to discriminate between such antigen and one or more reference antigen(s), since binding specificity is not an absolute, but a relative property. In its most general form (and when no defined reference is mentioned), “specific binding” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard color development (e.g. secondary antibody with horseradish peroxide and tetramethyl benzidine with hydrogenperoxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like.

However, “specific binding” also may refer to the ability of an antibody to discriminate between the target antigen and one or more closely related antigen(s), which are used as reference points, e.g. between human Alk1, murine Alk1, human Alk4 and murine Alk5. Additionally, “specific binding” may relate to the ability of an antibody to discriminate between different parts of its target antigen, e.g. different domains or regions of Alk1, such as epitopes in the N-terminal or in the C-terminal region of Alk1, or between one or more key amino acid residues or stretches of amino acid residues of Alk1.

Also, as used herein, an “immunoglobulin” (Ig) hereby is defined as a protein belonging to the class IgG, IgM, IgE, IgA, or IgD (or any subclass thereof), and includes all conventionally known antibodies and functional fragments thereof. A “functional fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of immunoglobulins for use in the present invention is IgG. “Functional fragments” of the invention include the domain of a F(ab′)₂ fragment, a Fab fragment and scFv. The F(ab′)₂ or Fab may be engineered to minimize or completely remove the intermolecular disulphide interactions that occur between the C_{H1} and C_{L} domains.

An antibody of the invention may be derived from a recombinant antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; Rothe et al., J. Mol. Biol. (2008) 376:1182-1200; and U.S. Pat. No. 6,300,064 issued to Knappik et al., which hereby are incorporated by reference in their entirety.

The present invention also provides isolated human or humanized antibody or functional fragment thereof comprising an antigen-binding region that is specific for Alk1 (SEQ ID NO: 193), wherein said antibody or functional fragment thereof posesses one or more of the following properties: a) is able to inhibit proliferation of endothelial cells, b) produce specific staining of tumor blood vessels in IHC, c) inhibit heterodimerization of Alk1 receptor d) inhibit BMP9 stimulated induction of SMAD7 expression in endothelial cells, e) inhibit BMP9 stimulated SMAD1/5/8 phosphorylation f) possess a specific affinity (Kd) down to or below 1 nM as determined by BIACORE or SET, g) inhibit angiogenesis, h) inhibit tumor growth, i) inhibit endothelial cell migration, j) inhibit smooth muscle cell proliferation, k) inhibit endothelial cell tube formation, l) induces ADCC in vivo.

In certain embodiments the present invention provides isolated human or humanized antibody, or functional fragments thereof, that are able to inhibit proliferation of endothelial cells.

In certain embodiments the present invention provides isolated human or humanized antibody, or functional fragments thereof, that produce specific staining of tumor blood vessels in IHC.

In certain embodiments the present invention provides isolated human or humanized antibody, or functional fragments thereof, that inhibit heterodimerization of Alk1 receptor.

In certain embodiments the present invention provides isolated human or humanized antibody, or functional fragments thereof, that inhibit BMP9 stimulated induction of SMAD7 expression in endothelial cells.

In certain embodiments the present invention provides isolated human or humanized antibody, or functional fragments thereof, that inhibit BMP9 stimulated SMAD1/5/8 phosphorylation.

In certain embodiments the present invention provides isolated human or humanized antibody, or functional fragments thereof, that possess a specific affinity (Kd) down to or below 1 nM as determined by BIACORE or SET. In certain preferred embodiments the antibodies, or functional fragments thereof, show a KD of less than 10 nM as measured on immobilized human Alk-1Fc protein in a BIACORE assay, as described herein. In other preferred embodiments the antibodies, or functional fragments thereof, show a KD of less than 5 nM as measured on immobilized human Alk-1Fc protein in a BIACORE assay, as described herein. In yet other preferred embodiments the antibodies, or functional fragments thereof, show a KD of less than 50 nM as measured on immobilized murine Alk-1Fc protein in a BIACORE assay, as described herein. In certain preferred embodiments the antibodies, or functional fragments thereof, show a KD of less than 200 pM as measured on with human Alk-1Fc protein in a SET assay, as described herein. In other preferred embodiments the antibodies, or functional fragments thereof, show a KD of less than 20 pM as measured on with human Alk-1Fc protein in a SET assay, as described herein. In even more preferred embodiments the antibodies, or functional fragments thereof, show a KD of less than 10 pM as measured on with human Alk-1Fc protein in a SET assay, as described herein.

In certain embodiments the present invention provides isolated human or humanized antibody, or functional fragments thereof, that inhibit angiogenesis.

In certain embodiments the present invention provides isolated human or humanized antibody, or functional fragments thereof, that inhibit tumor growth.

In certain embodiments the present invention provides isolated human or humanized antibody, or functional fragments thereof, that inhibit endothelial cell migration.

In certain embodiments the present invention provides isolated human or humanized antibody, or functional fragments thereof, that inhibit smooth muscle cell proliferation.

In certain embodiments the present invention provides isolated human or humanized antibody, or functional fragments thereof, that inhibit endothelial cell tube formation.

In certain embodiments the present invention provides isolated human or humanized antibody, or functional fragments thereof, that induce ADCC in vivo.

In certain preferred embodiments, the present invention also provides antibodies, or functional fragments thereof, that compete for binding to the epitopes of the antibodies of the present invention.

In certain embodiments the present invention provides antibodies, or functional fragments thereof, that compete for binding to the epitope of an antibody, or functional fragments thereof, which comprises an H-CDR3 region depicted in SEQ ID NOS: 49-64 or is coded by SEQ ID NOS: 1-16.

In certain embodiments the present invention provides antibodies, or functional fragments thereof, that compete for binding to the epitope of an antibody, or functional fragments thereof, which comprises an H-CDR2 region depicted in SEQ ID NOS: 65-80, or is coded by SEQ ID NOS:17-32 or 194-261.

In certain embodiments the present invention provides antibodies, or functional fragments thereof, that compete for binding to the epitope of an antibody, or functional fragments thereof, which comprises an H-CDR1 region depicted in SEQ ID NOS: 81-96 or is coded by SEQ ID NOS: 33-48.

In certain embodiments the present invention provides antibodies, or functional fragments thereof, that compete for binding to the epitope of an antibody, or functional fragments thereof, which comprises an L-CDR3 region depicted in SEQ ID NOS: 145-160, or is coded by SEQ ID NOS: 97-112 or 262-329.

In certain embodiments the present invention provides antibodies, or functional fragments thereof, that compete for binding to the epitope of an antibody, or functional fragments thereof, which comprises an L-CDR1 region depicted in SEQ ID NOS: 177-192 or is coded by SEQ ID NOS: 129-144.

In certain embodiments the present invention provides antibodies, or functional fragments thereof, that compete for binding to the epitope of an antibody, or functional fragments thereof, which comprises an L-CDR2 region depicted in in SEQ ID NOS: 161-176 or is coded by SEQ ID NOS: 113-128.

In certain embodiments the present invention provides antibodies, or functional fragments thereof, that compete for binding to the epitope of an antibody, or functional fragments thereof, which comprises a heavy chain amino acid sequence selected from the group consisting of (i) SEQ ID NOS: 49-96 and 228-261, and (ii) a sequence having at least 60 percent sequence identity in the CDR regions with the CDR regions depicted in SEQ ID NOS: 49-96 and/or 228-261.

In certain embodiments the present invention provides antibodies, or functional fragments thereof, that compete for binding to the epitope of an antibody, or functional fragments thereof, which comprises a light chain amino acid sequence selected from the group consisting of (i) SEQ ID NOS: 145-192 and 296-329; and (ii) a sequence having at least 60 percent sequence identity in the CDR regions with the CDR regions depicted in SEQ ID NOS: 145-192 and 296-329.

All the above properties, to the best of our knowledge, have never been described before and constitute novel and surprising features of the antibodies of the present invention.

### Antibodies of the Invention

Throughout this document, reference is made to the following representative antibodies of the invention: MOR05369, MOR05370, MOR05371, MOR05372, MOR05373, MOR05374, MOR05375, MOR05376, MOR05377, MOR05440, MOR05441, MOR05442, MOR05444, MOR05445, MOR05447, MOR05448 hereafter referred as “parental” antibodies, represents an antibody having a variable heavy chain region corresponding to SEQ ID NOS: 1-48 (DNA)/SEQ ID NOS: 49-96 (peptide) and a variable light chain region corresponding to SEQ ID NO: 97-144 (DNA)/SEQ ID NOS: 145-192 (peptide).

Furthermore, throughout this document, reference is made to the following representative antibodies of the invention: MOR06315, MOR06316, MOR06321, MOR06322, MOR06323, MOR06324, MOR06336, MOR06452, MOR06311, MOR06312, MOR06333, MOR06338, MOR06360, MOR06444, MOR06446, MOR06445, MOR06447, MOR06448, MOR06449, MOR06331, MOR06332, MOR06320, MOR06346, MOR06450, MOR06451, MOR06325, MOR06337, MOR06376, MOR06317, MOR06318, MOR06319, MOR06326, MOR06335, MOR06313, MOR06314 hereafter referred as “affinity maturated”, or “maturated” antibodies, represents an antibody having a variable heavy chain region corresponding to SEQ ID NOS: 194-227 (DNA)/SEQ ID NOS: 228-261 (peptide) and a variable light chain region corresponding to SEQ ID NO: 262-295 (DNA)/SEQ ID NOS: 296-329 (peptide).

Tables 1 and 2 provide a summary of affinities of representative parental and maturated antibodies of the invention, as determined by surface plasmon resonance (Biacore; table 1) and solution equilibrium titration (SET; table 2).

TABLE 1
Parental antibody affinities determined by Biacore

K_{D} (nM) (n = 1)

| MOR0# | huALK1-Fc | muALK1-Fc |
|---|---|---|
| 5369 | 7.4 | 23 |
| 5370 | 7.7 | nd |
| 5371 | 12.3 | nd |
| 5372 | 4.0 | nd |
| 5373 | 9.0 | nd |
| 5374 | 14.1 | nd |
| 5375 | 131.9 | nd |
| 5376 | 12.7 | nd |
| 5377 | 24.5 | no binding |
| 5440 | 7.4 | 93 |
| 5441 | 5.6 | nd |
| 5442 | 18.8 | nd |
| 5444 | 19.9 | nd |
| 5445 | 26.4 | 30 |
| 5447 | 5.9 | no binding |
| 5448 | 4.6 | 39 |

TABLE 2
Maturated antibody affinities determined by SET

| | Kd [pM] | | |
|---|---|---|---|
| | huALK1- | muALK1- | |
| | Fc | Fc | n= |
| MOR05377 | 24660 | 16000 | 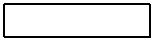 |
| MOR06311 | 102.5 | low | 2 |
| MOR06312 | 160 | 2000 | 1 |
| MOR06333 | 5.6 | 1200 | 3 |
| MOR06338 | 20.5 | nd | 4 |
| MOR06360 | 40.6 | 51000 | 3 |
| MOR06347 | 24 | nd | 3 |
| MOR05448 | 36896 | low | 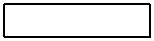 |
| MOR06314 | 63.5 | 4700 | 2 |
| MOR06313 | 160 | 2100 | 1 |
| MOR05445 | 52785 | 11000 | 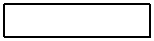 |
| MOR06326 | 30 | 21000 | 3 |
| MOR06335 | 29.25 | 4000 | 4 |
| MOR05372 | 36369 | — | 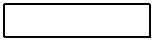 |
| MOR06337 | 52 | nd | 1 |
| MOR06323 | 226 | low | 1 |
| MOR05376 | 47324 | — | 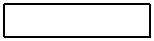 |
| MOR06317 | 162 | — | 2 |
| MOR06318 | 220 | — | 2 |
| MOR06319 | 104 | — | 2 |
| MOR06334 | 5.5 | — | 2 |
| MOR05444 | 22133 | — | 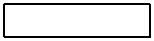 |
| MOR06320 | 265 | — | 1 |
| MOR06331 | 5.5 | — | 2 |
| MOR06332 | 5.5 | — | 2 |
| MOR06346 | nd | — | 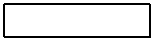 |
| MOR05369 | 72164 | 80600 | 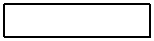 |
| MOR06315 | 172 | low | 1 |
| MOR06316 | 411.5 | low | 2 |
| MOR06321 | 149 | 13000 | 1 |
| MOR06322 | 210 | 8700 | 1 |
| MOR06323 | 226 | low | 1 |
| MOR06324 | 234 | 16700 | 1 |
| MOR06336 | 8.5 | nd | 2 |

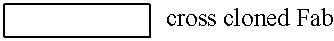

Most affinity matured binders showed K_{D} values below 200 pM. The best Fabs reached affinities in the 5-20 pM range in SET.

With reference to Table 1 and 2, the affinity of antibodies was measured by surface plasmon resonance (Biacore) on immobilized recombinant human and murine Alk1-Fc or by solution equilibrium titration (SET).

The Biacore studies were performed on directly immobilized antigen. The Fab format of antibodies exhibit an monovalent affinity range between about 131 and 4 nM on immobilized Alk1-Fc protein with MOR05372 showing the highest affinity, followed by MOR05448. Eight out of 16 Fab fragments had a KD below 10 nM. In particular, MOR05372 and MOR05448 had KD values below 5 nM. In addition for 4/16 Fab fragments KDs on mouse ALK1-Fc could be not determined.

Another feature of preferred antibodies of the invention is their specificity for an area within the N-terminal extracellular region of Alk1. For example, MOR 5444 of the invention can bind specifically to the N-terminal region of Alk1.

An antibody of the invention preferably is species cross-reactive with humans and at least one other species, which may be a mouse or a rat. An antibody that is cross reactive with at least one other species, for example, can provide greater flexibility and benefits over known anti-Alk1 antibodies, for purposes of conducting in vivo studies in multiple species with the same antibody.

Cross reactivity was tested with purified Fab fragments by ELISA on human and murine ALK1 as well as on human ALK4 and murine ALK5. No cross reactivity to human ALK4 and murine ALK5 was seen as summarized in table 3.

Preferably, an antibody of the invention not only is able to bind to Alk1, but also is able to block its function. More specifically, an antibody of the invention can mediate its therapeutic effect by Alk1 via antibody-effector functions.

TABLE 3
Cross reactivity of parental and maturated Fabs to murine
ALK1, human ALK4 and murine ALK5 measured by ELISA

ELISA

| MOR nb | huALK1 | mALK1 | huALK4 | mALK5 |
|---|---|---|---|---|
| 5369 | + | (+/−) | − | − |
| 6315 | + | + | − | − |
| 6316 | + | + | − | − |
| 6321 | + | + | − | − |
| 6322 | + | + | − | − |
| 6323 | + | + | − | − |
| 6324 | + | + | − | − |
| 6336 | + | + | − | − |
| 6452 (15x22) | + | + | − | − |
| 5377 | + | + | − | − |
| 6311 | + | + | − | − |
| 6312 | + | + | − | − |
| 6333 | + | + | − | − |
| 6338 | + | + | − | − |
| 6360 | + | + | − | − |
| 6444 (11x38) | + | + | − | − |
| 6446 (12x38) | + | (+/−) | − | − |
| 6445 (33x38) | + | − | − | − |
| 6447 (11x60) | + | + | − | − |
| 6448 (12x60) | + | + | − | − |
| 6449 (33x60) | + | + | − | − |
| 5444 | + | − | − | − |
| 6331 | + | − | − | − |
| 6332 | + | − | − | − |
| 6320 | + | − | − | − |
| 6346 | + | − | − | − |
| 6450 (31x20) | + | − | − | − |
| 6451 (32x30) | + | − | − | − |
| 5372 | + | − | − | − |
| 6325 | + | + | − | − |
| 6337 | + | + | − | − |
| 5376 | + | − | − | − |
| 6317 | + | + | − | − |
| 6318 | + | + | − | − |
| 6319 | + | − | − | − |
| 6334 | + | − | − | − |
| 5445 | + | + | − | − |
| 6326 | + | − | − | − |
| 6335 | + | + | − | − |
| 5448 | + | + | − | − |
| 6313 | + | + | − | − |
| 6314 | + | + | − | − |

### Peptide Variants

Antibodies of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating that variants having the ability to block function fall within the scope of the present invention.

A variant can include, for example, an antibody that has at least one altered complementarity determining region (CDR) (hyper-variable) and/or framework (FR) (variable) domain/position, vis-à-vis a peptide sequence disclosed herein. To better illustrate this concept, a brief description of antibody structure follows.

An antibody is composed of two peptide chains, each containing one (light chain) or three (heavy chain) constant domains and a variable region (VL, VH), the latter of which is in each case made up of four FR regions and three interspaced CDRs. The antigen-binding site is formed by one or more CDRs, yet the FR regions provide the structural framework for the CDRs and, hence, play an important role in antigen binding. By altering one or more amino acid residues in a CDR or FR region, the skilled worker routinely can generate mutated or diversified antibody sequences, which can be screened against the antigen, for new or improved properties, for example.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophan, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in a-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

As used herein, “sequence identity” between two polypeptide sequences, indicates the percentage of amino acids that are identical between the sequences. “Sequence homology” indicates the percentage of amino acids that either are identical or that represent conservative amino acid substitutions. Preferred polypeptide sequences of the invention have a sequence identity in the CDR regions of at least 60%, more preferably, at least 70% or 80%, still more preferably at least 90% and most preferably at least 95%. Preferred antibodies also have a sequence homology in the CDR regions of at least 80%, more preferably 90% and most preferably 95%.

DNA molecules of the Invention

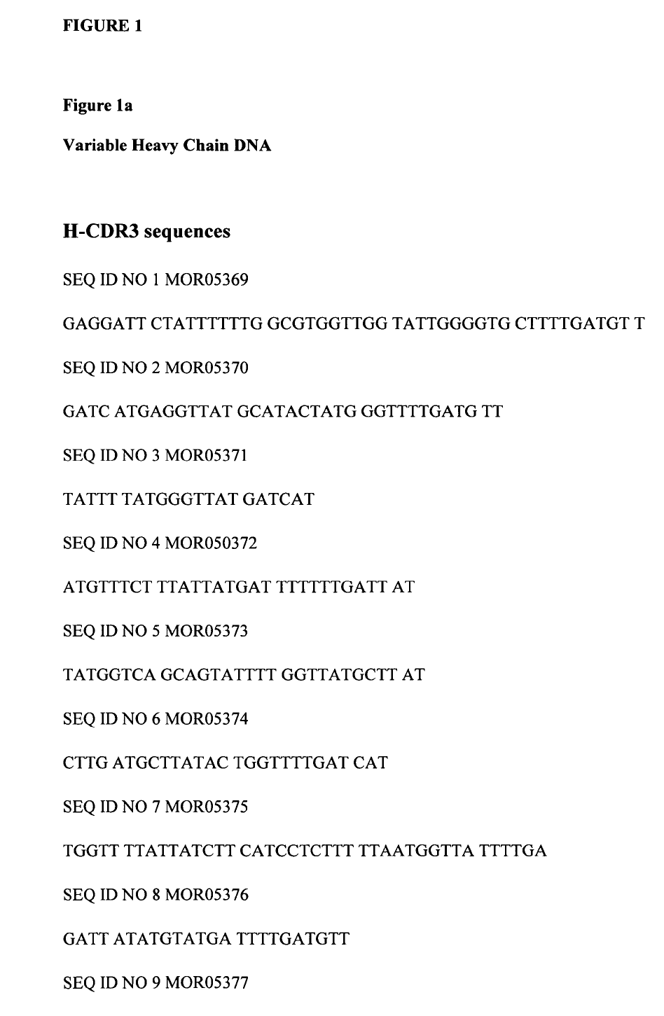

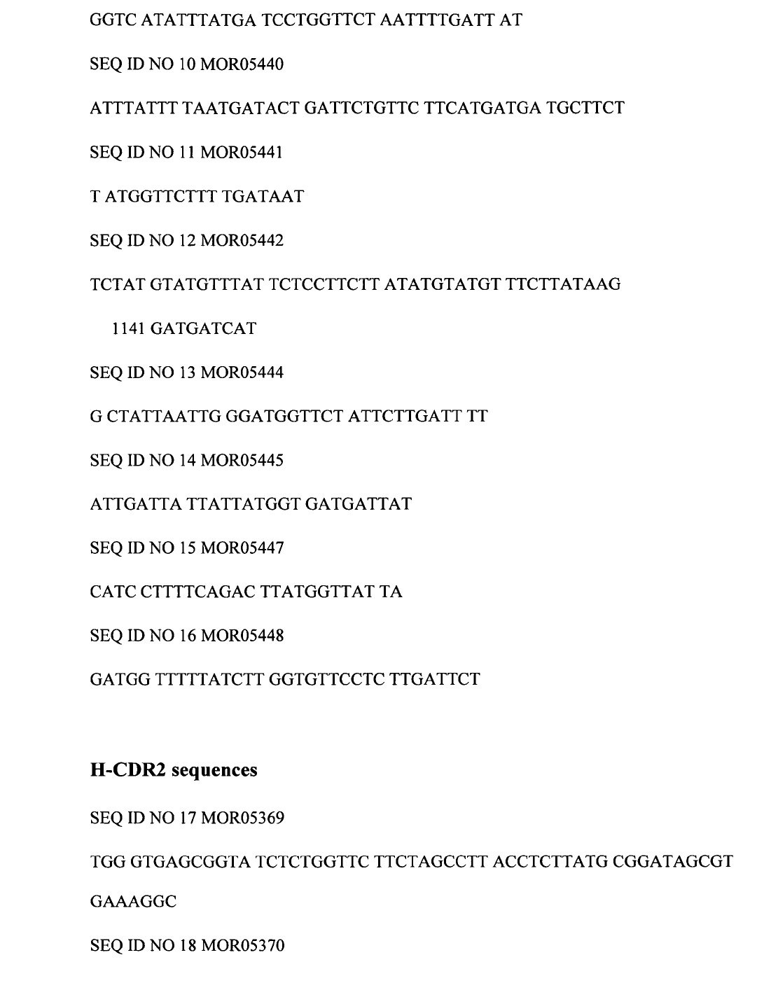

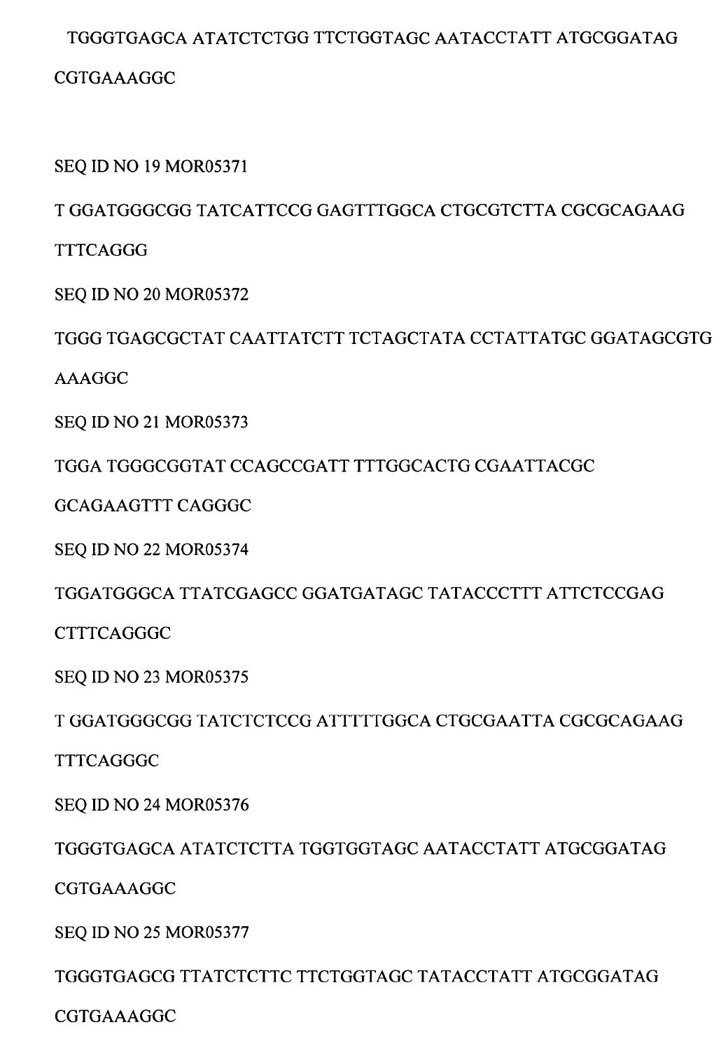

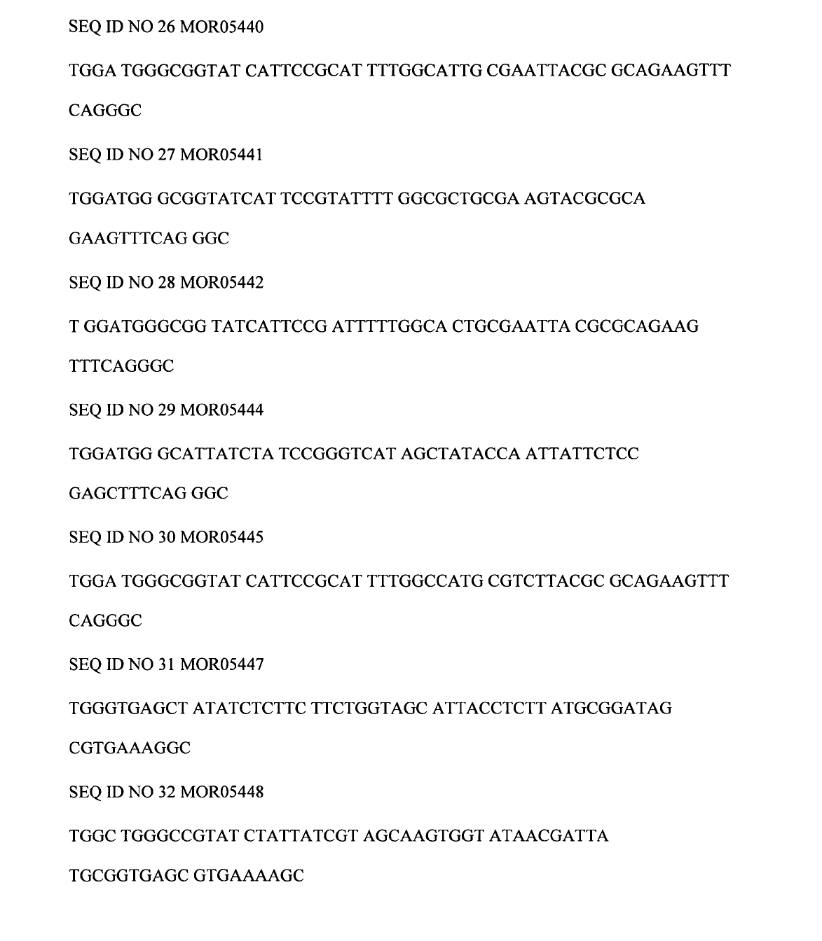

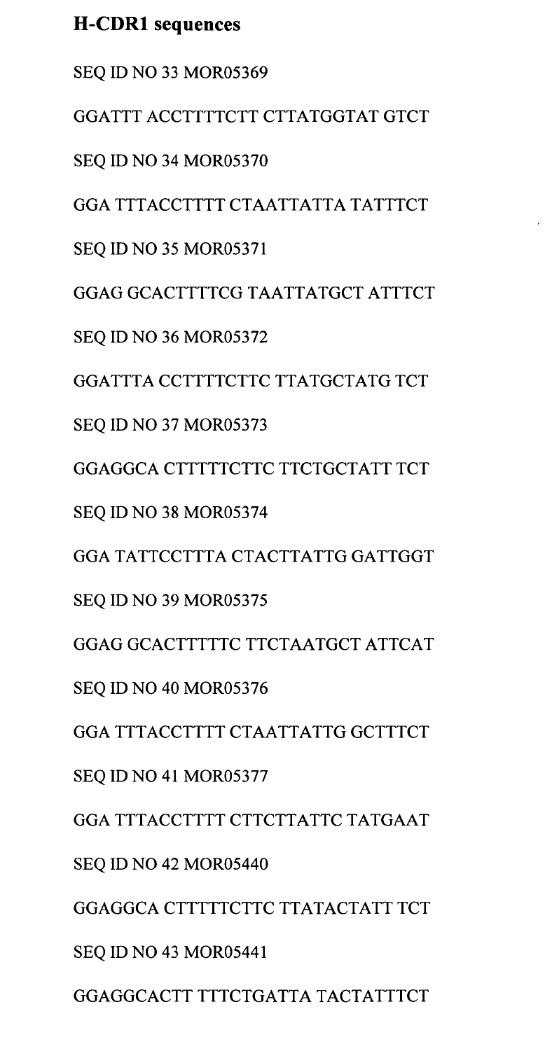

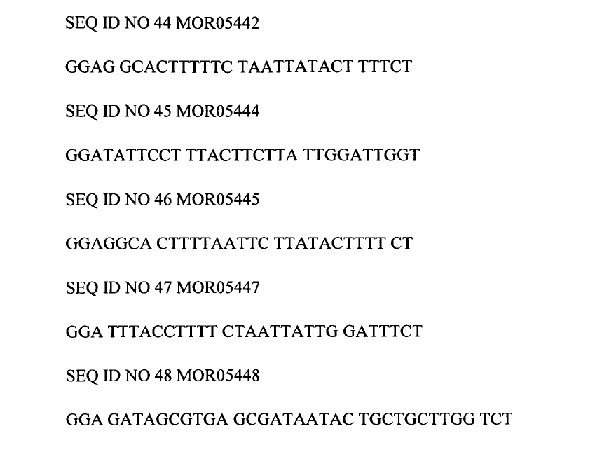

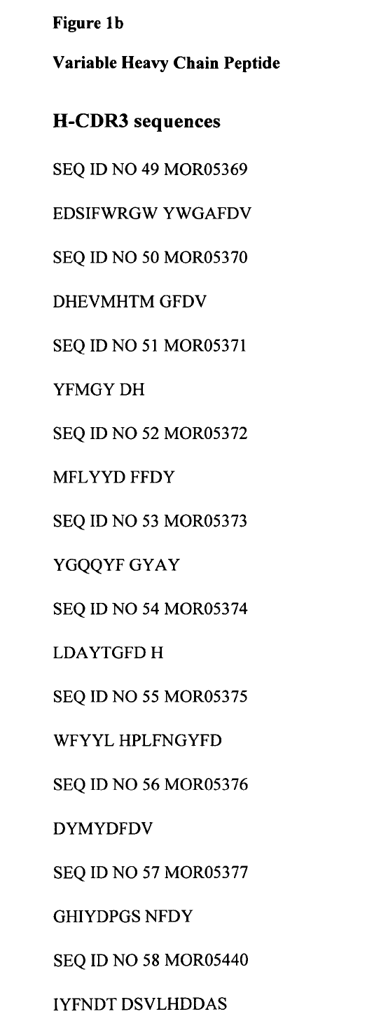

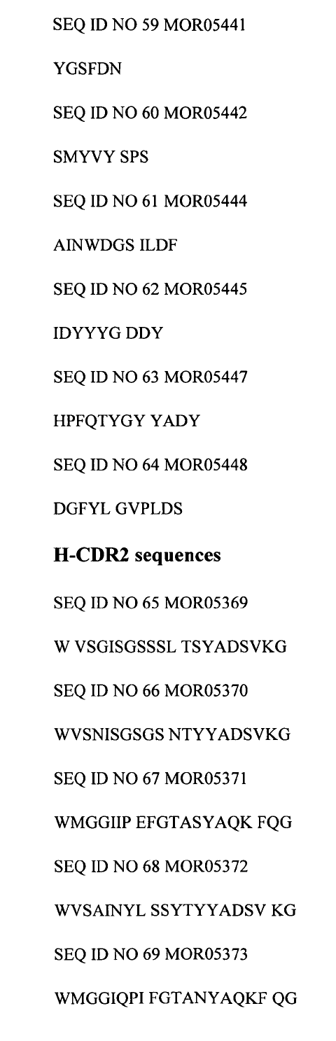

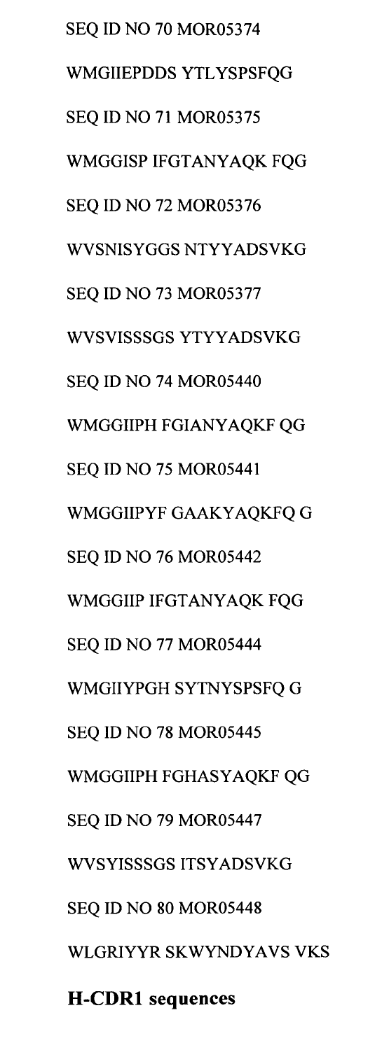

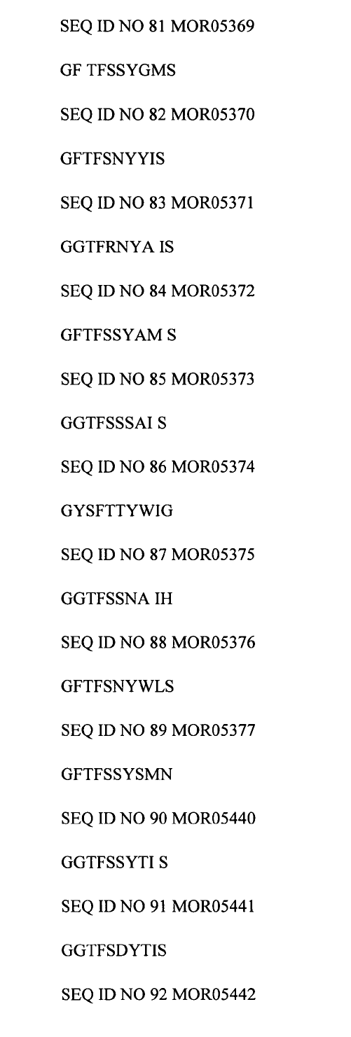

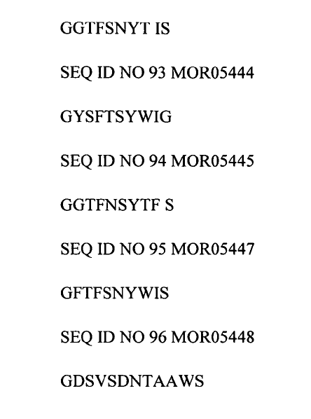

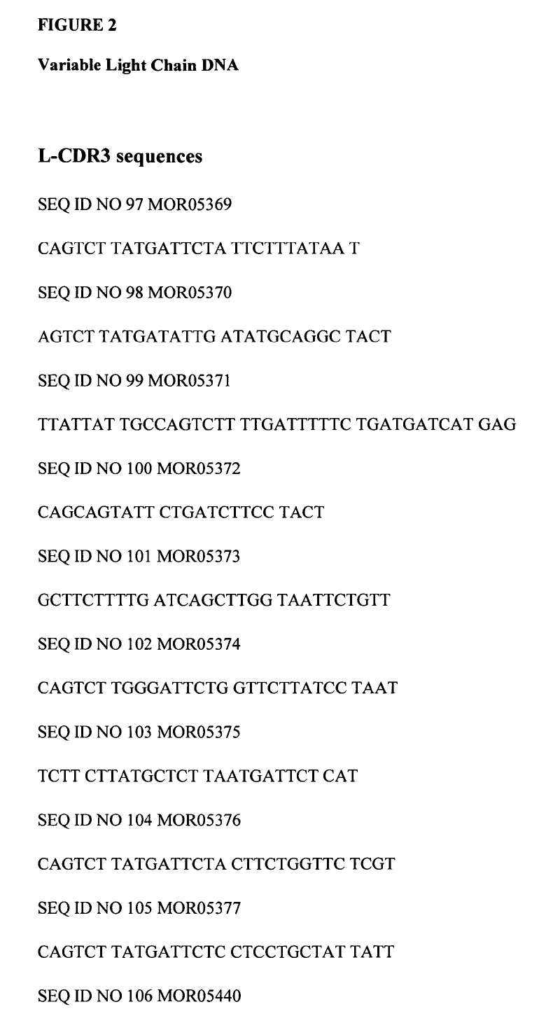

The present invention also relates to the DNA molecules that encode an antibody of the invention. These sequences include, but are not limited to, those DNA molecules set forth in FIGS. 1a and 2a.

DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 (Sambrook, J., Fritsch, E. F. and Maniatis, T. (1989) Molecular Cloning: A laboratory manual, Cold Spring Harbor Laboratory Press, Cold Spring Harbor, USA) and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):
a. Tm=2° C.(A+T)+4° C.(G+C)
b. Another familiar equation for DNA which is valid for oligos longer than 50 nucleotides from pH 5 to 9 is: Tm=81.5+16.6 log M+41(XG+XC)−500/L -0.62F, where M is the molar concentration of monovalent cations, XG and XC are the mole fractions of G and C in the oligo, L is the length of the shortest strand in the duplex, and F is the molar concentration of formamide.
c. Tm decreases by about 1° C. for every 1% of mismatched base pairs.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

First, in the binding phase, the probe is bound to the target under conditions favoring hybridization. Stringency is usually controlled at this stage by altering the temperature. For high stringency, the temperature is usually between 65° C. and 70° C., unless short (<20 nt) oligonucleotide probes are used. A representative hybridization solution comprises 6×SSC, 0.5% SDS, 5×Denhardt's solution and 100 μg of nonspecific carrier DNA. See Ausubel et al., section 2.9, supplement 27 (1994). Of course, many different, yet functionally equivalent, buffer conditions are known. Where the degree of relatedness is lower, a lower temperature may be chosen. Low stringency binding temperatures are between about 25° C. and 40° C. Medium stringency is between at least about 40° C. to less than about 65° C. High stringency is at least about 65° C.

Second, the excess probe is removed by washing. It is at this phase that more stringent conditions usually are applied. Hence, it is this “washing” stage that is most important in determining relatedness via hybridization. Washing solutions typically contain lower salt concentrations. One exemplary medium stringency solution contains 2×SSC and 0.1% SDS. A high stringency wash solution contains the equivalent (in ionic strength) of less than about 0.2×SSC, with a preferred stringent solution containing about 0.1×SSC. The temperatures associated with various stringencies are the same as discussed above for “binding.” The washing solution also typically is replaced a number of times during washing. For example, typical high stringency washing conditions comprise washing twice for 30 minutes at 55° C. and three times for 15 minutes at 60° C.

Accordingly, the present invention includes nucleic acid molecules that hybridize to the molecules of set forth in FIGS. 1a and 2a under high stringency binding and washing conditions, where such nucleic molecules encode an antibody or functional fragment thereof having properties as described herein. Preferred molecules (from an mRNA perspective) are those that have at least 75% or 80% (preferably at least 85%, more preferably at least 90% and most preferably at least 95%) homology or sequence identity with one of the DNA molecules described herein. In one particular example of a variant of the invention, nucleic acid position 7 in SEQ ID NOS: can be substituted from a C to a G, thereby changing the codon from CAA to GAA.

### Functionally Equivalent Variants

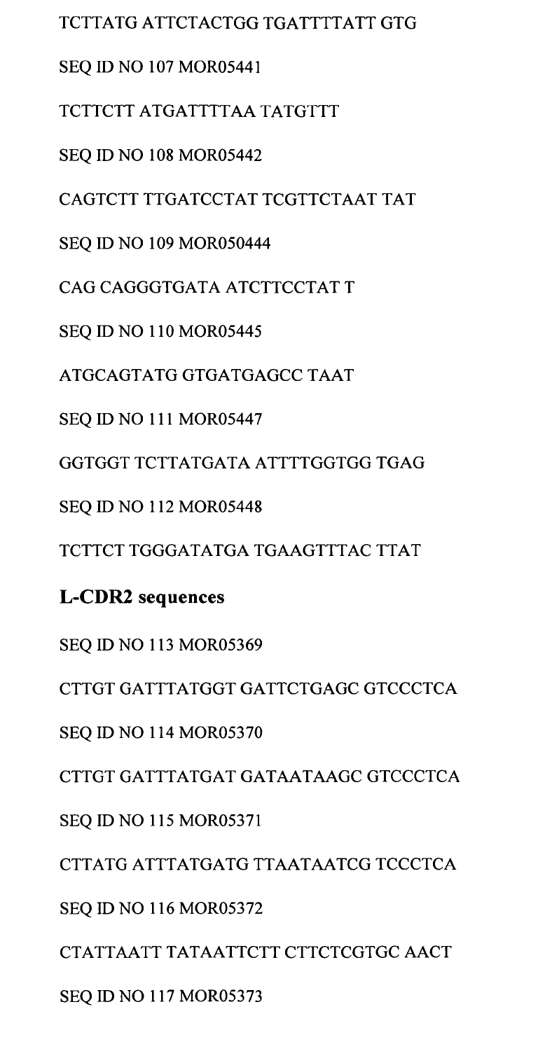

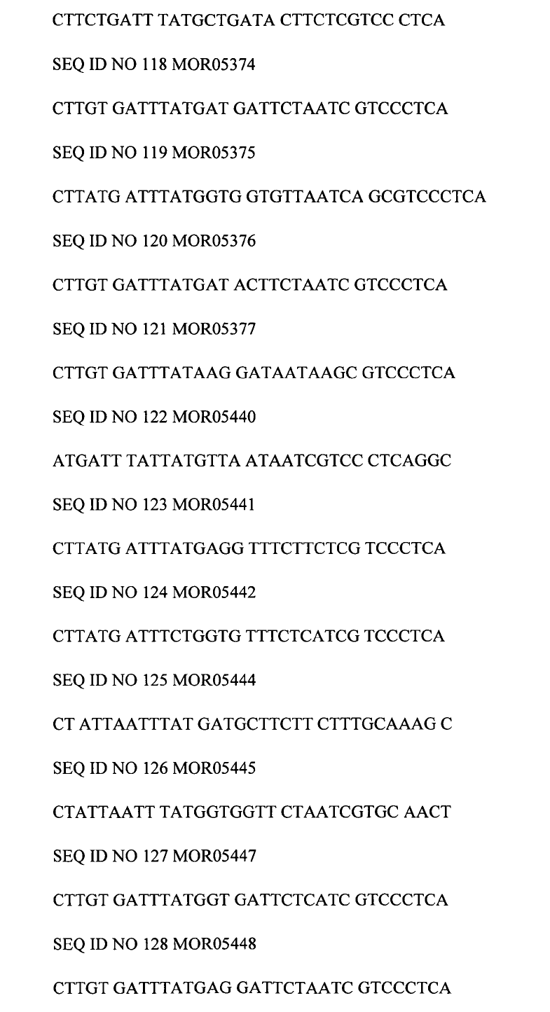

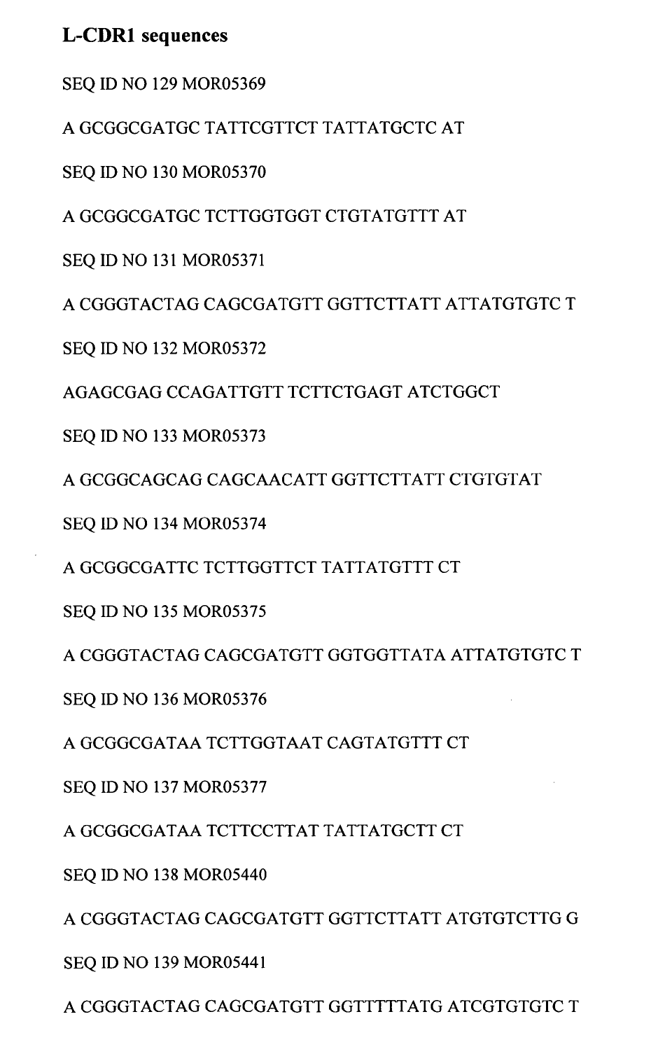

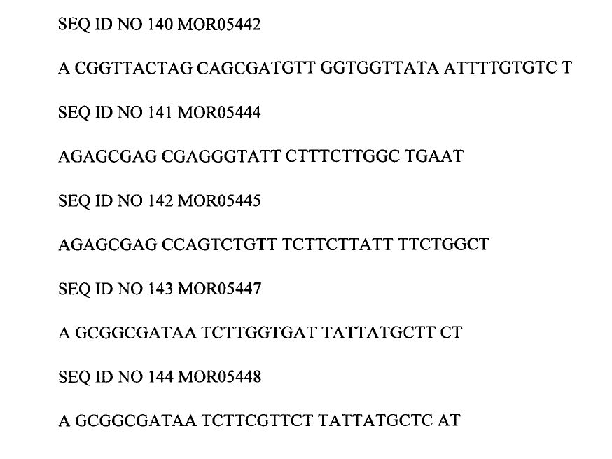

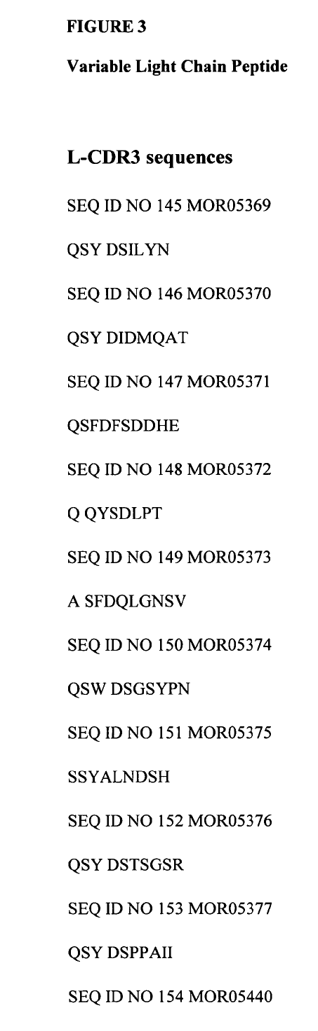

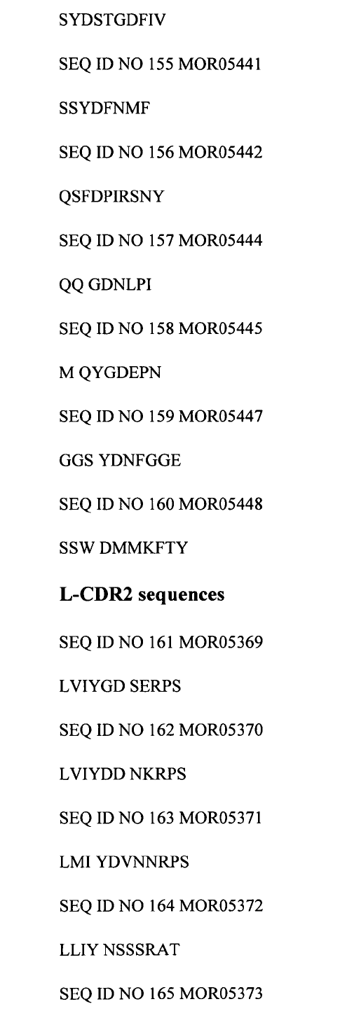

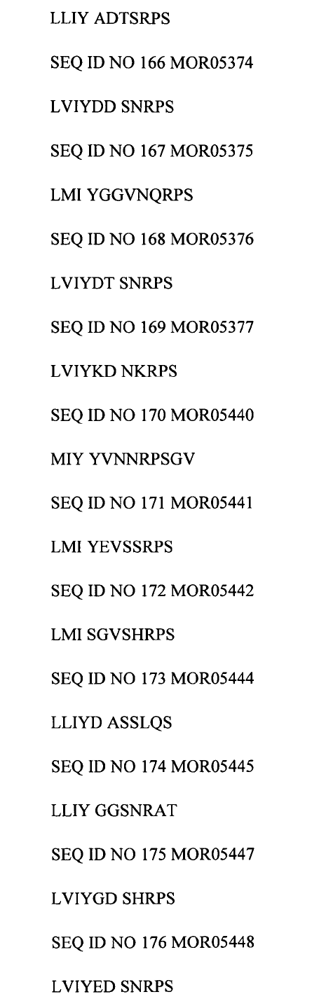

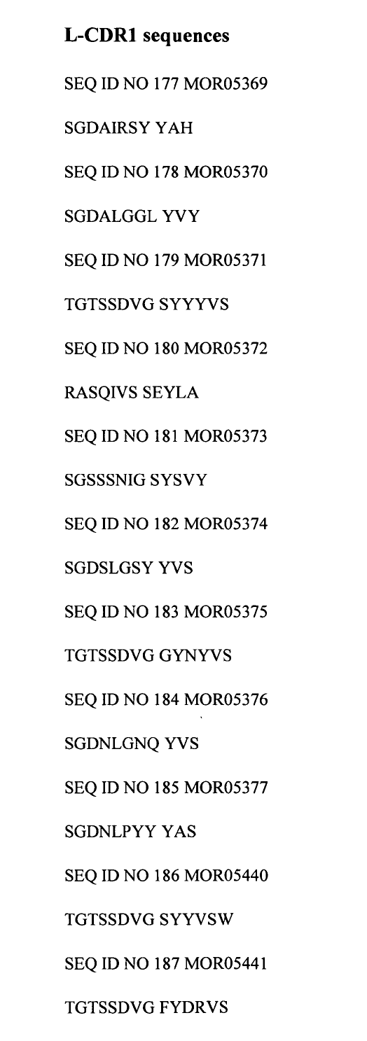

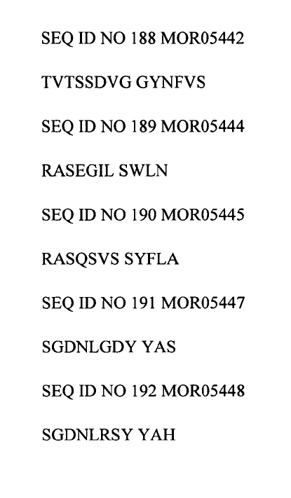

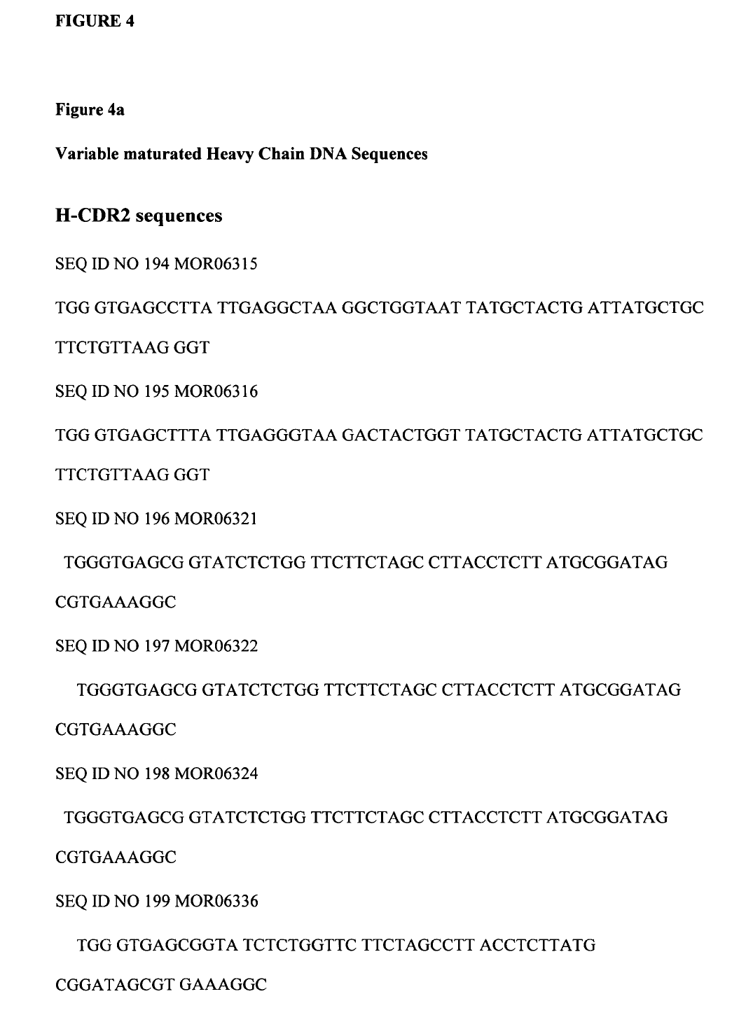

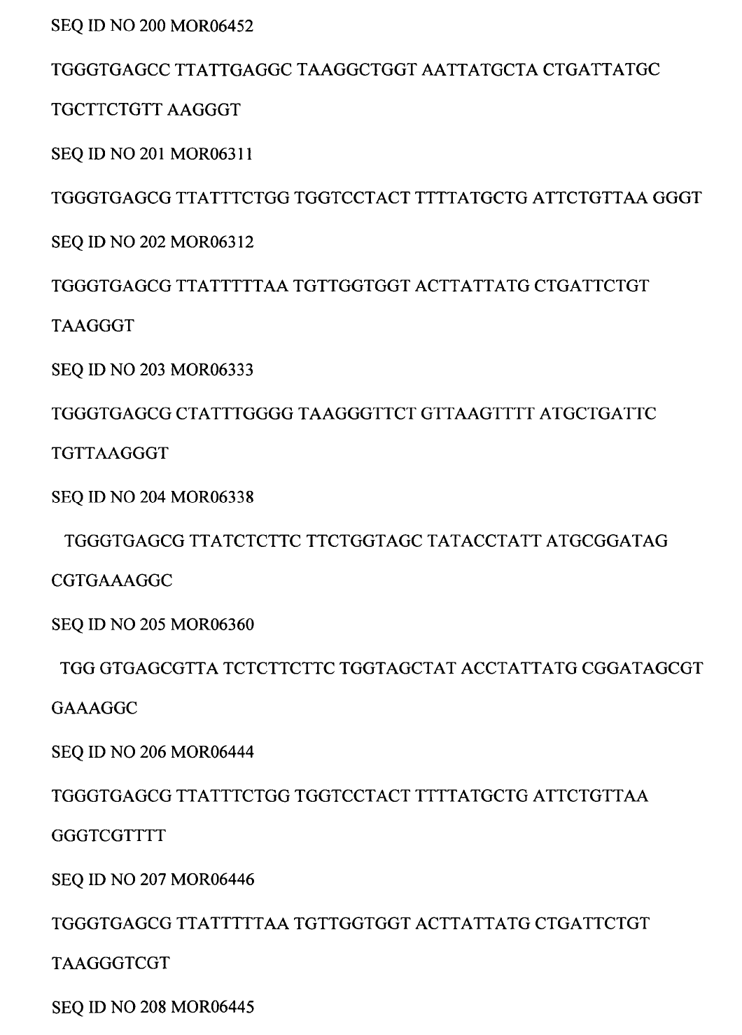

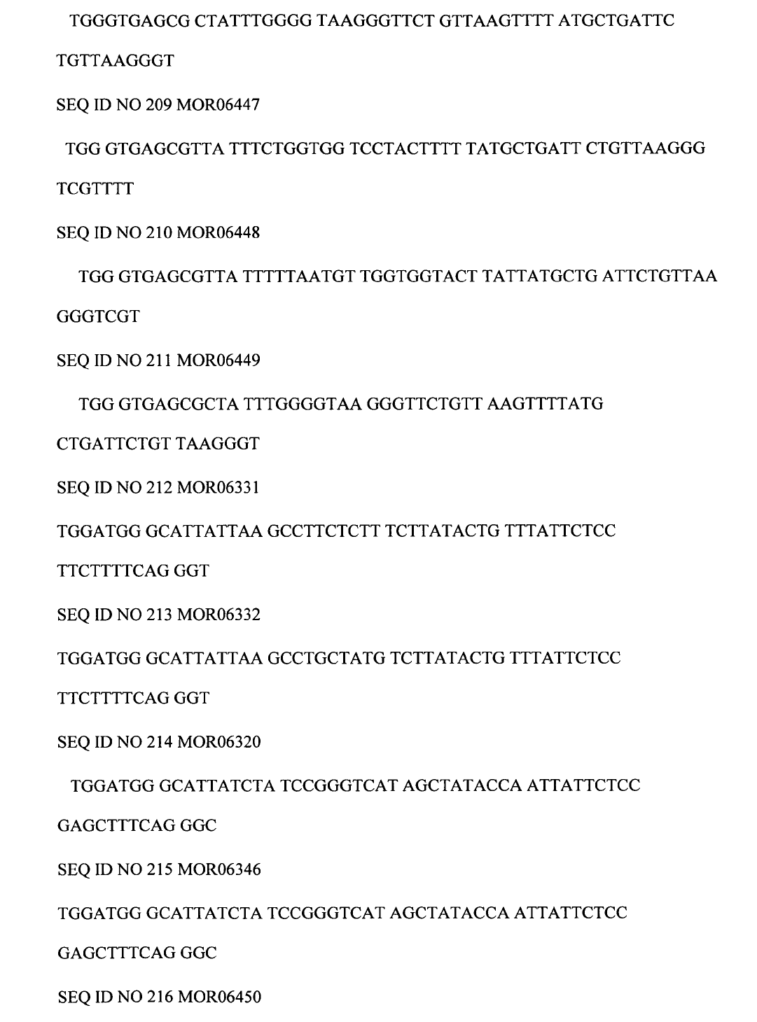

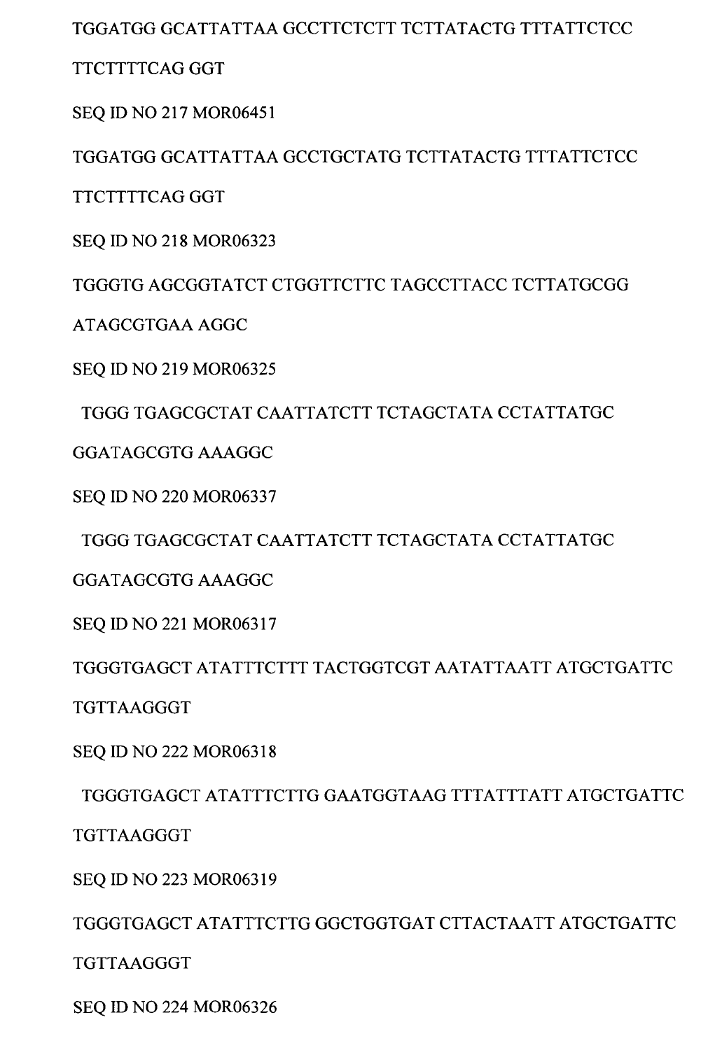

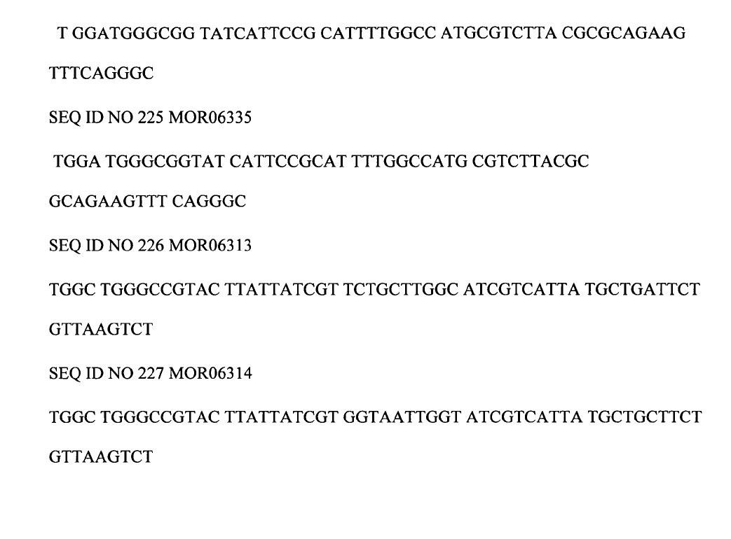

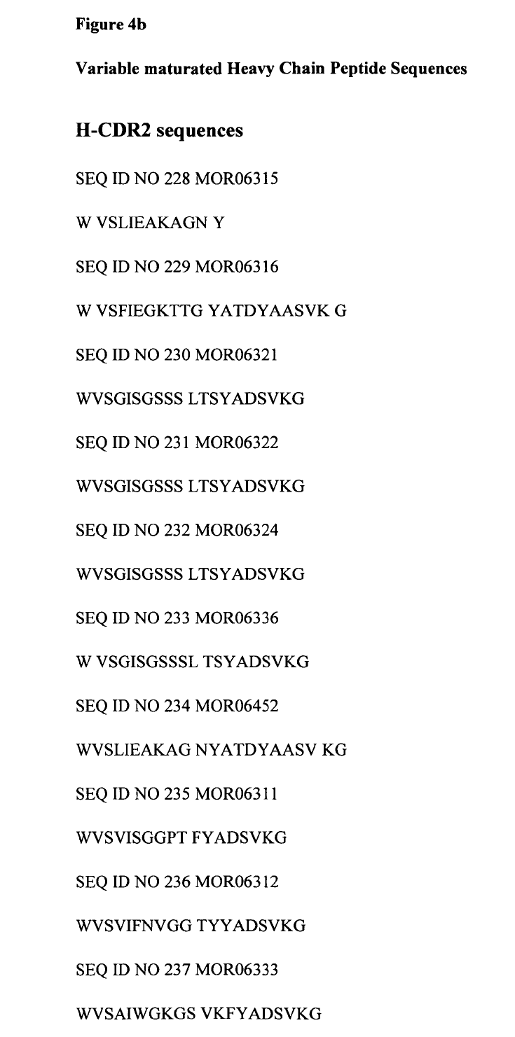

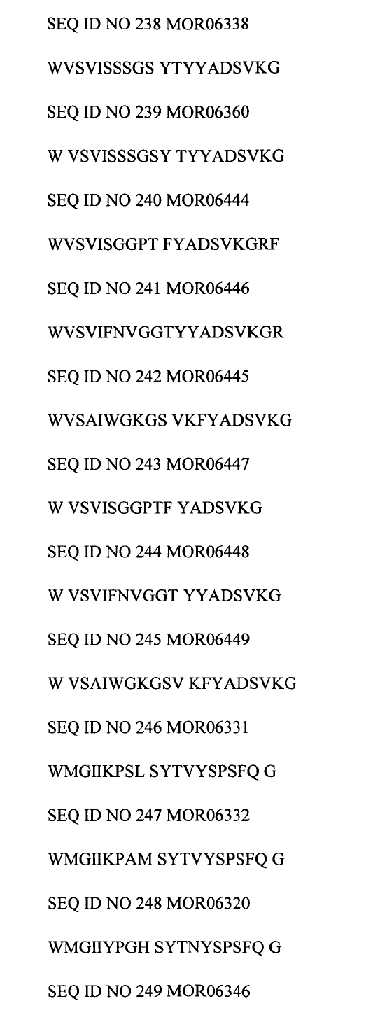

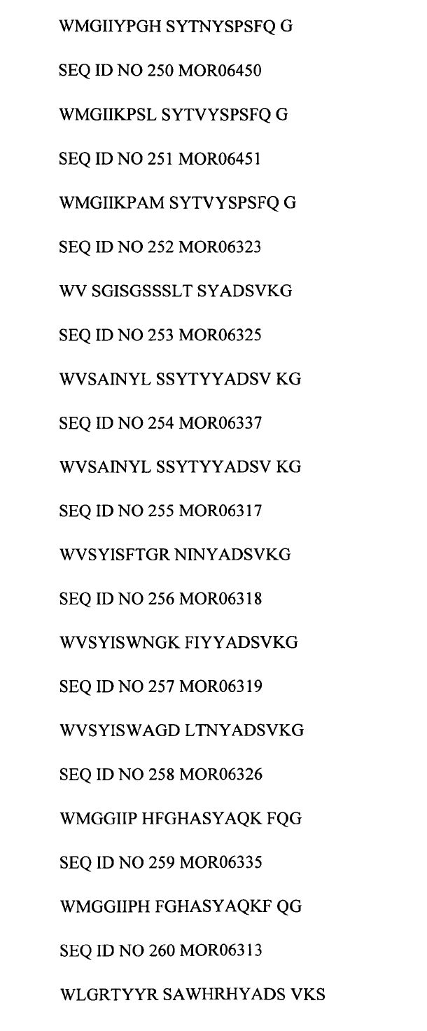

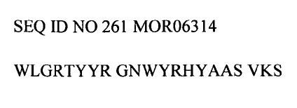

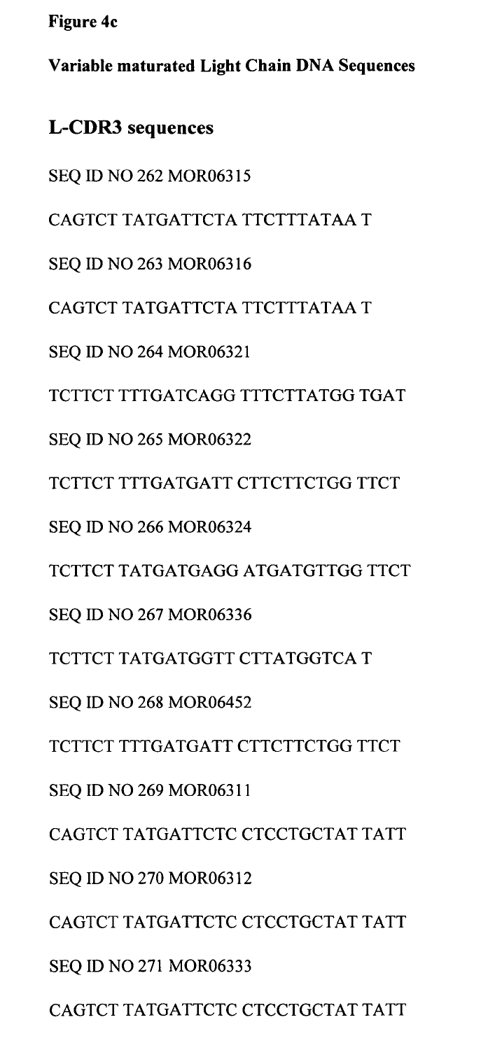

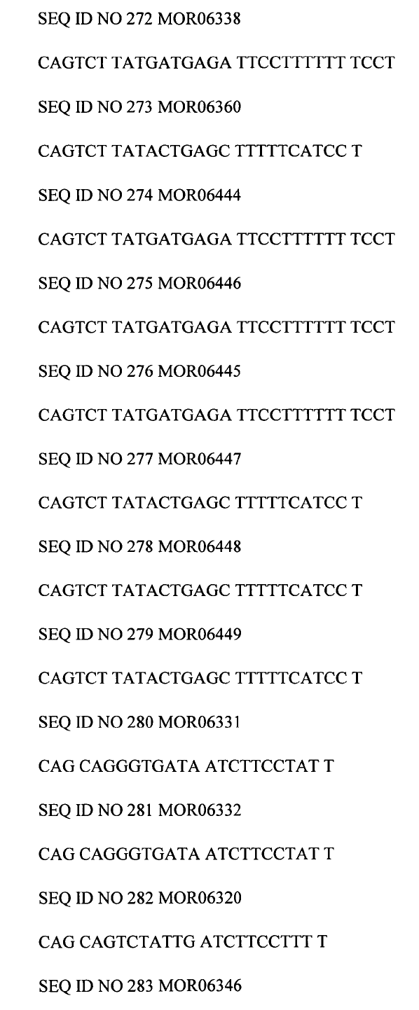

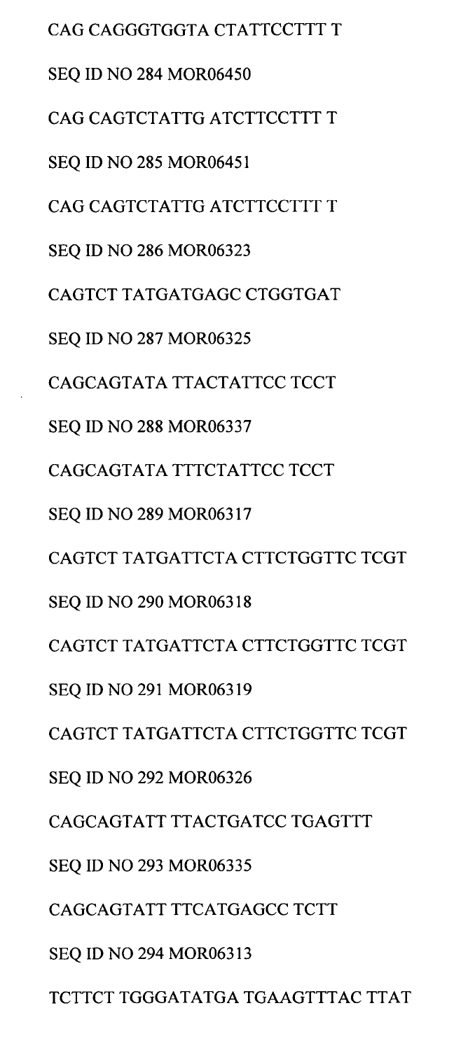

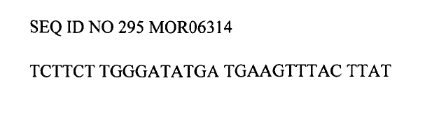

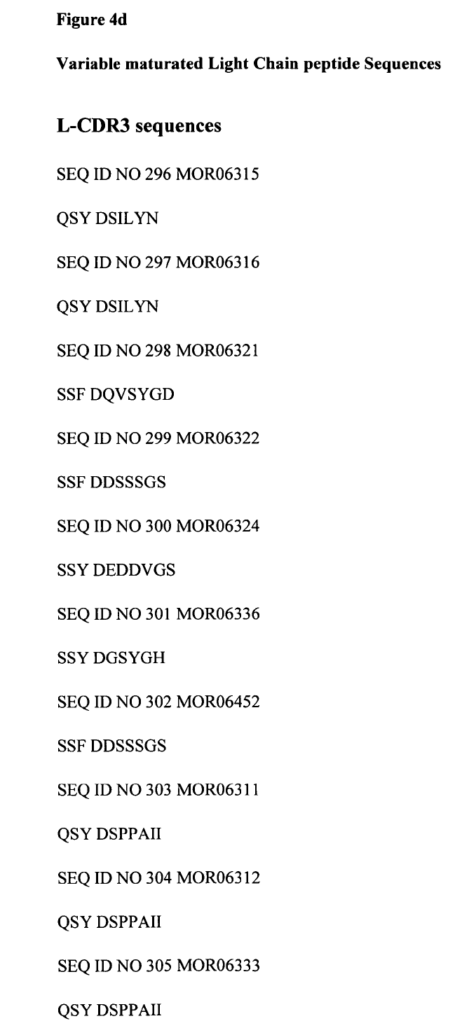

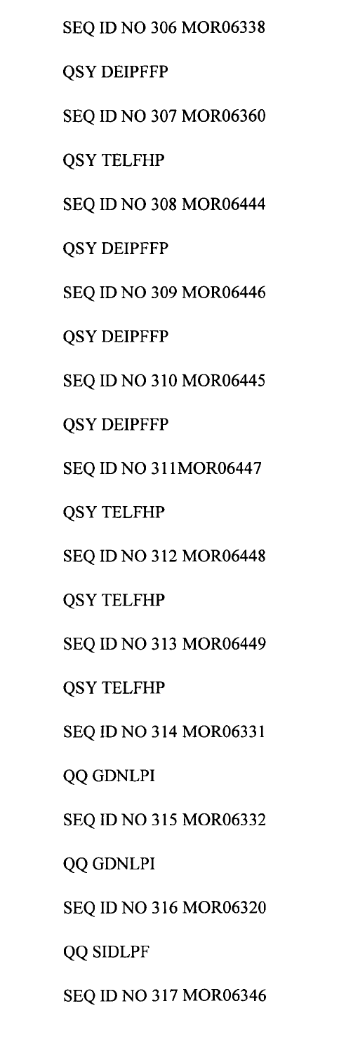

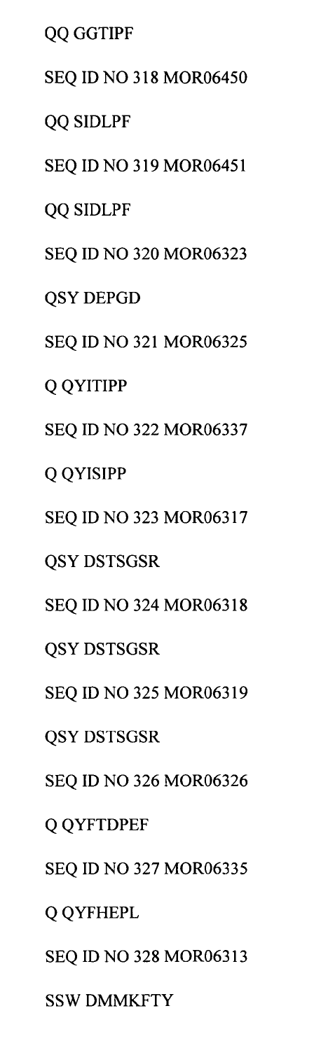

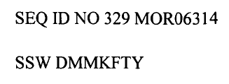

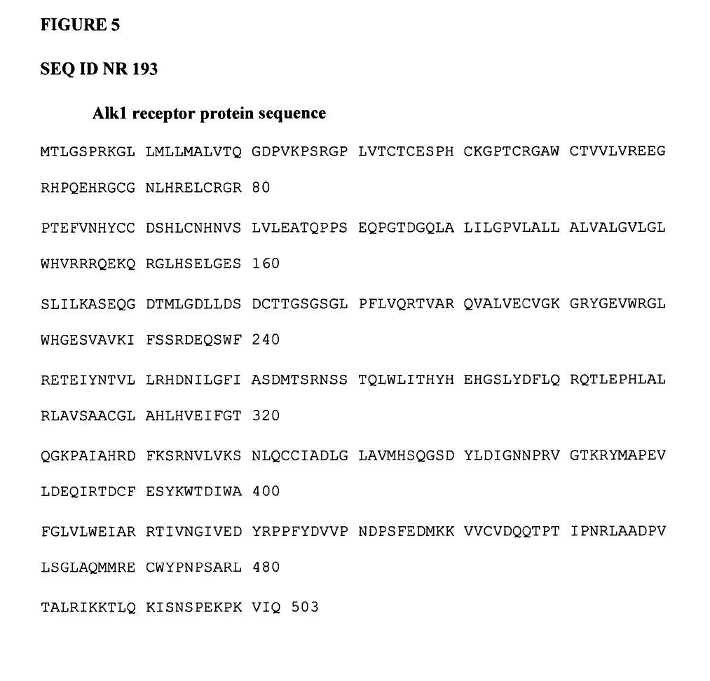

Yet another class of DNA variants within the scope of the invention may be described with reference to the product they encode (see the peptides listed in FIGS. 1b and 3). These functionally equivalent genes are characterized by the fact that they encode the same peptide sequences found in FIGS. 1b and 3 due to the degeneracy of the genetic code.

It is recognized that variants of DNA molecules provided herein can be constructed in several different ways. For example, they may be constructed as completely synthetic DNAs. Methods of efficiently synthesizing oligonucleotides in the range of 20 to about 150 nucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present invention further provides recombinant DNA constructs comprising one or more of the nucleotide sequences of the present invention. The recombinant constructs of the present invention are used in connection with a vector, such as a plasmid, phagemid, phage or viral vector, into which a DNA molecule encoding an antibody of the invention is inserted.

The encoded gene may be produced by techniques described in Sambrook et al., 1989, and Ausubel et al., 1989. Alternatively, the DNA sequences may be chemically synthesized using, for example, synthesizers. See, for example, the techniques described in OLIGONUCLEOTIDE SYNTHESIS (1984, Gait, ed., IRL Press, Oxford), which is incorporated by reference herein in its entirety. Recombinant constructs of the invention are comprised with expression vectors that are capable of expressing the RNA and/or protein products of the encoded DNA(s). The vector may further comprise regulatory sequences, including a promoter operably linked to the open reading frame (ORF). The vector may further comprise a selectable marker sequence. Specific initiation and bacterial secretory signals also may be required for efficient translation of inserted target gene coding sequences.

The present invention further provides host cells containing at least one of the DNAs of the present invention. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant construct into the host cell can be effected by calcium phosphate transfection, DEAE, dextran mediated transfection, electroporation or phage infection.

### Bacterial Expression

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and bacterial origin of replication derived from commercially available plasmids typically containing elements of the well known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

### Therapeutic Methods

Therapeutic methods involve administering to a subject in need of treatment a therapeutically effective amount of an antibody contemplated by the invention. A “therapeutically effective” amount hereby is defined as the amount of an antibody that is of sufficient quantity to block Alk1 activity in a treated cells or an area of a subject—either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, monkey or other lower-order primate).

An antibody of the invention might be co-administered with known medicaments, and in some instances the antibody might itself be modified. For example, an antibody could be conjugated to an immunotoxin or radioisotope to potentially further increase efficacy.

The inventive antibodies can be used as a therapeutic or a diagnostic tool in a variety of situations where Alk1 is undesirably expressed or found. Disorders and conditions particularly suitable for treatment with an antibody of the inventions are conditions associated with pathogenic angiogenesis such as cancer or macular degeneration.

To treat any of the foregoing disorders, pharmaceutical compositions for use in accordance with the present invention may be formulated in a conventional manner using one or more physiologically acceptable carriers or excipients. An antibody of the invention can be administered by any suitable means, which can vary, depending on the type of disorder being treated. Possible administration routes include parenteral (e.g., intramuscular, intravenous, intraarterial, intraperitoneal, or subcutaneous), intrapulmonary and intranasal, and, if desired for local immunosuppressive treatment, intralesional administration. In addition, an antibody of the invention might be administered by pulse infusion, with, e.g., declining doses of the antibody. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered. The skilled artisan will recognize that the route of administration will vary depending on the disorder or condition to be treated.

Determining a therapeutically effective amount of the novel polypeptide, according to this invention, largely will depend on particular patient characteristics, route of administration, and the nature of the disorder being treated. General guidance can be found, for example, in the publications of the International Conference on Harmonisation and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

### Diagnostic Methods

Alk1 is highly expressed on endothelial cells in certain malignancies; thus, an anti-Alk1 antibody of the invention may be employed in order to image or visualize a site of possible Alk1 activation in a patient. In this regard, an antibody can be detectably labeled, through the use of radioisotopes, affinity labels (such as biotin, avidin, etc.) fluorescent labels, paramagnetic atoms, etc. Procedures for accomplishing such labeling are well known to the art. Clinical application of antibodies in diagnostic imaging are reviewed by Grossman, H. B., Urol. Clin. North Amer. 13:465-474 (1986)), Unger, E. C. et al., Invest. Radiol. 20:693-700 (1985)), and Khaw, B. A. et al., Science 209:295-297 (1980)).

### Therapeutic And Diagnostic Compositions

The antibodies of the present invention can be formulated according to known methods to prepare pharmaceutically useful compositions, wherein an antibody of the invention (including any functional fragment thereof) is combined in a mixture with a pharmaceutically acceptable carrier vehicle. Suitable vehicles and their formulation are described, for example, in REMINGTON'S PHARMACEUTICAL SCIENCES (18th ed., Alfonso R. Gennaro, Ed., Easton, Pa.: Mack Pub. Co., 1990). In order to form a pharmaceutically acceptable composition suitable for effective administration, such compositions will contain an effective amount of one or more of the antibodies of the present invention, together with a suitable amount of carrier vehicle.

Preparations may be suitably formulated to give controlled-release of the active compound. Controlled-release preparations may be achieved through the use of polymers to complex or absorb anti-Alk1 antibody. The controlled delivery may be exercised by selecting appropriate macromolecules (for example polyesters, polyamino acids, polyvinyl, pyrrolidone, ethylenevinyl-acetate, methylcellulose, carboxymethylcellulose, or protamine, sulfate) and the concentration of macromolecules as well as the methods of incorporation in order to control release. Another possible method to control the duration of action by controlled release preparations is to incorporate anti-Alk1 antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980).

The compounds may be formulated for parenteral administration by injection, e.g., by bolus injection or continuous infusion. Formulations for injection may be presented in unit dosage form, e.g., in ampules, or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

The invention further is understood by reference to the following working examples, which are intended to illustrate and, hence, not limit the invention.

### EXAMPLES

### Example 1

### Antibody Generation from HuCAL Libraries

For the generation of therapeutic antibodies against Alk1, selections with the MorphoSys HuCAL GOLD® phage display library were carried out. HuCAL GOLD® is a Fab library based on the HuCAL® concept (Knappik et al., 2000; Krebs et al., 2001), in which all six CDRs are diversified, and which employs the CysDisplay® technology for linking Fab fragments to the phage surface (Löhning, 2001; Rothe et al., 2008).

### A. Phagemid Rescue, Phage Amplification and Purification

HuCAL GOLD® phagemid library was amplified in 2×TY medium containing 34 μg/ml chloramphenicol and 1% glucose (2×TY-CG). After helper phage infection (VCSM13) at an OD600 of 0.5 (30 min at 37° C. without shaking; 30 min at 37° C. shaking at 250 rpm), cells were spun down (4120 g; 5 min; 4° C.), resuspended in 2×TY/34 μg/ml chloramphenicol/50 μg/ml kanamycin and grown overnight at 22° C. Phages were PEG-precipitated from the supernatant, resuspended in PBS/20% glycerol and stored at −80° C. Phage amplification between two panning rounds was conducted as follows: mid-log phase TG1 cells were infected with eluted phages and plated onto LB-agar supplemented with 1% of glucose and 34 μg/ml of chloramphenicol (LB-CG). After overnight incubation at 30° C., colonies were scraped off, adjusted to an OD600 of 0.5 and helper phage added as described above.

B. Pannings with HuCAL GOLD®

For the selections HuCAL GOLD® antibody-phages were divided into three pools corresponding to different VH master genes (pool 1: VH1/5λκ, pool 2: VH3 λκ, pool 3: VH2/4/6 λκ). These pools were individually subjected to 3 rounds of whole cell panning on Alk1-expressing X cells followed by pH-elution and a post-adsorption step on Alk1-negative X-cells for depletion of irrelevant antibody-phages. Finally, the remaining antibody phages were used to infect E. coli TG1 cells. After centrifugation the bacterial pellet was resuspended in 2×TY medium, plated on agar plates and incubated overnight at 30° C. The selected clones were then scraped from the plates, phages were rescued and amplified. The second and the third round of selections were performed as the initial one.

The Fab encoding inserts of the selected HuCAL GOLD® phages were subcloned into the expression vector pMORPH®x9_Fab_FH (Rauchenberger et al., 2003) to facilitate rapid expression of soluble Fab. The DNA of the selected clones was digested with XbaI and EcoRI thereby cutting out the Fab encoding insert (ompA-VLCL and phoA-Fd), and cloned into the XbaI/EcoRI cut vector pMORPH®x9_Fab_FH. Fab expressed in this vector carry two C-terminal tags (FLAG™ and His6 tag) for detection and purification.

### Example 2

### Expression and Purification of HuCAL-Fab Antibodies in E. coli

Expression of Fab fragments encoded by pMORPHX9 FH in E. coli TG-1 F-cells was carried out in shaker flask cultures with 1 l of 2×YT medium supplemented with 34 μg/ml chloramphenicol. After induction with 0.5 mM IPTG, cells were grown at 30° C. for 20 h. Cells were lysed and Fab fragments isolated by HT-IMAC-purification. Protein homogeneity and monomeric state was determined by size exclusion chromatography (SEC). Concentrations were determined by UV-spectrophotometry.

### Example 3

### Cloning, Expression and Purification of HuCAL® IgG1

In order to express full length IgG, variable domain fragments of heavy (VH) and light chains (VL) were subcloned from Fab expression vectors into appropriate pMORPH2®_hIg vectors (see Figures). Restriction endonuclease pairs BlpI/MfeI (insert-preparation) and BlpI/EcoRI (vector-preparation) were used for subcloning of the VH domain fragment into pMORPH2®_hIgG1. Enzyme-pairs EcoRV/HpaI (lambda-insert) and EcoRV/BsiWI (kappa-insert) were used for subcloning of the VL domain fragment into the respective pMORPH2®_hIgκ_{—}1 or pMORPH2®_h_Igλ_{—}1 vectors. Resulting IgG constructs were expressed in HKB11 cells by transient transfection procedures. IgGs were purified from cell culture supernatants by affinity chromatography via Protein A Sepharose column. Further down stream processing included a buffer exchange by gel filtration and sterile filtration of purified IgG. Quality control revealed a purity of >90% by reducing SDS-PAGE and >90% monomeric IgG as determined by analytical size exclusion chromatography. The endotoxin content of the material was determined by a kinetic LAL based assay (Cambrex European Endotoxin Testing Service, Belgium).

### Example 4

### ELISA Analysis

Antigen binding properties of selected Fab lysates were assessed by ELISA on purified human and murine Alk1-Fc, human A1k4-Fc and murine Alk5-Fc.Cross reactivity to huAlk4-Fc and muAlk5-Fc was observed very rarely. A high number of Fab fragments cross-reactive to mouse Alk1 were selected from the panning including mouse Alk1-Fc in the second round, indicating the validity of this panning approach.

Table 3 summarises the cross-reactivity of parental and maturated binders to human Alk1, murine Alk1, human Alk4 and murine Alk5, as measured by ELISA. Strikingly, most of the human Alk1 specific binders were cross reactive with murine Alk1, but none of the binders showed any crossreactivity with human Alk4 or murine Alk5.

### Example 5

### Bioveris Screening

Biotinylated Alk1-Fc was captured on streptavidin magnetic beads and incubated with diluted Fab containing E. coli lysates. Antigen specific signals were measured applying the Bioveris electrochemiluminescence (ECL) system using an anti-h Fab-BV tagged secondary antibody. Increased ECL values indicates an improved affinity but does not allow direct calculation of KDs. 1920 clones were screened and about 500 binders with good ECL values derived from the 6 panning pools were picked and transferred into 6×96 well compression plates for further analysis. Fabs with the highest ECL values were purified and subjected to affinity measurement by solution equilibrium titration (SET; Haenel et al, 2005; Example 7) and surface plasmon resonance (Biacore) (see Example 6)

### Example 6

### Surface Plasmon Resonance

The kinetic constants kₒₙ and k_{off} were determined with serial dilutions of the respective Fab binding to Alk1-Fc fusion protein captured on a sensor chip using the BIAcore 3000 instrument (Biacore, Uppsala, Sweden). For antigen capture a CM5 senor chip (Biacore) was coated with goat-anti-human-Fc antibody (Dianova) using standard EDC-NHS amine coupling chemistry (immobilization of approximately 8000 RU). Alk1-fc was captured by injecting 20 μL of a 100 nM Alk1-Fc solution. On the reference flow cell which was coated with the goat anti-human-Fc antibody, no antigen was captured. Kinetic measurements were done in PBS (136 mM NaCl, 2.7 mM KCl, 10 mM Na2HPO4, 1.76 mM KH2PO4 pH 7.4) at a flow rate of 20 μl/min using Fab concentration range from 15.6-500 nM. Injection time for each concentration was 1 min, followed by 2 min dissociation phase. For regeneration two injections of 5 μl 10 mM Glycine/HCl were used. All sensograms were fitted locally using BIA evaluation software 3.2 (Biacore).

Results are summarized in Table 1. Binders showed an affinity range from between 133 and 4 nM on immobilized human Alk-1Fc protein. Most of the binders showed a KD of less than 10 nM, and numerous binders even showed a KD of less than 5 nM. Binders also showed a KD of less than 50 nM measured with immobilized murine Alk-1Fc protein.

Biacore kₒₙ and k_{off} curve fits for the Fab binder MOR06326 are shown in FIG. 7.

### Example 7

### KD Determination by Solution Equilibrium Titration (SET)

Affinity determination of anti ALK1 Fab fragments was also preformed by solution equilibrium titration generally as previously described (Haenel et al. 2005). Fab fragments were applied in a constant concentration and mixed with serial dilutions of Alk1-Fc. After over night incubation at room temperature (establishement of Fab-ALk1-Fc equilibrium) M-280 Streptavidin Dynabeads (Dynal) and By-tag® (BioVeris) labelled goat-anti-human Fab antibody were added. The portion of free antibody was thereby captured by incubation with the antigen coupled beads. Electrochemiluminescence signals were detected by an M-384 Analyzer (BioVeris). Data evaluation was done utilizing XLfit 4.1 software (IDBS).

Results are summarized in Table 2. Most of the binders showed a KD of less than 200 pM, and numerous binders showed a KD of less than 20 pM or even less than 10 pM, as measured with human Alk-1Fc protein.

### Example 8

### Affinity Maturation

It was decided to proceed with H-CDR2 and L-CDR3 maturation of a large number of parental Fabs: 12 ALK1 specific Fab fragments, including MOR 5444 and MOR 5377 which were IHC positive were selected for maturation. Selection was based on binding affinity, crossreactivity to muALK1 and positive staining in IHC. Twelve parental Fab were chosen for affinity maturation: MOR05444, 5377, 5445, 5448, 5369, 5441, 5442, 5370, 5371, 5372, 5373 and 5376.

For each of the 12 selected parental Fab fragments maturation libraries diversified in LCDR3 and HCDR2, respectively were prepared, resulting in 24 different maturation libraries.

Maturation libraries were pooled on the phage level in six different phage pools before initiation of the pannings HCDR-2 and LCDR-3 libraries were kept separately. The affinity maturation library phage pools were composed as follows:

L-CDR3 diversified
Pool 1: IHC positive: MOR05444, -5377
Pool 2: mouse Alk1 cross-reactive Fabs MOR05445, -5448 and -5369
Pool 3: human Alk1 specific Fabs MOR05441, -5442, -5370, -5371, -5372, -5373 and -5376.
   H-CDR2 diversified:
Pool 4: IHC positive: MOR05444, -5377
Pool 5: mouse Alk1 cross-reactive Fabs MOR05445, -5448 and -5369
Pool 6: human Alk1 specific Fabs MOR05441, -5442, -5370, -5371, -5372, -5373 and -5376.

### Example 9

### Solid Phase Pannings of Affinity Maturated Fabs

Solid phase pannings were performed as described in example 1, section B with the exception that the stringency conditions were increased. Coating was performed with lower concentrations of purified hALK1, i.e. 100 ng/ml in the first round, 20 ng/ml in the 2nd round and 5 ng/ml for the 3rd round. For the alternating pannings hALK1 was used at 100 ng/ml and 20 ng/ml for the 1^{st} and 3rd round, respectively, and mALK1 was used at 20 ng/ml for the 2nd round. Washing periods were also increased, including overnight washing in the 3rd round.

### Example 10

### Consolidation and Purification of Matured Fab Fragments

Based on the results of the Bioveris and ELISA screening, 96 clones were selected for sequencing. IHC and cross reactivity patterns of the respective parental Fabs were also taken into consideration for the selection. Sequence analysis showed that a large diversity was achieved since 67/96 clones were unique.

Based on the ECL values and sequencing results (CDR diversity) and based on phage pool origin (i.e. candidates from each pool were taken to maintain parental sequence diversity), 26 clones were chosen for expression and purification. These 26 clones were derived from 7 different parental Fab fragments. For MOR05444, MOR05369 and MOR05377 it was possible to select L-CDR3 as well as H-CDR2 improved clones leaving the option of cross cloning (see Example 11). 25 clones were expressed and purified.

### Example 11

### Cross Cloning

Affinity maturated derivatives of MOR05377, MOR05344 and MOR05369 (=parentals) were selected for cross cloning, based on their performance in affinity and bioactivity. Cross cloning was done by combining an affinity matured VL with an affinity matured VH derived from the same parental. The H-CDR2 improved clones were cut with XbaI/SphI, and the vector-fragment, including the optimized H-CDR2, was isolated and ligated with the VL fragment being optimized in LCDR3. Single clone expression and preparation of periplasmic extracts containing HuCAL®—Fab fragments were performed as described previously (Rauchenberger et al., 2003). The clones were verified by sequencing.

### Example 12

### Conversion from Monovalent Fab into the dHLX- and IgG1-Format

In order to express full length IgG, variable domain fragments of heavy (VH) and light chains (VL) were amplified by PCR and subcloned from Fab expression vectors into appropriate pMorph2®_hIg vectors for human IgG1. Restriction enzymes MfeI and BlpI were used for subcloning of the VH domain fragment into pMorph2®_h_IgG1f. EcoRV, BsiWI were used for subcloning of the Vkappa domain fragment into pMorph2®_h_Igκ and EcoRV, HpaI for subcloning of the Vlambda domain fragment into pMorph2®_h_Igλ2. In order to express bivalent Fab fragments, Fab fragement was excised via EcoRI/XbaI from the Fab expression vector pMx9_FH into pMx9_dHLX_MH. The encoded dHLX domain allows non-covalent association of two Fab molecules (Plückthun & Pack, 1997).

### Example 13

### Specificity Analysis by Immunohisto-Chemistry (IHC)

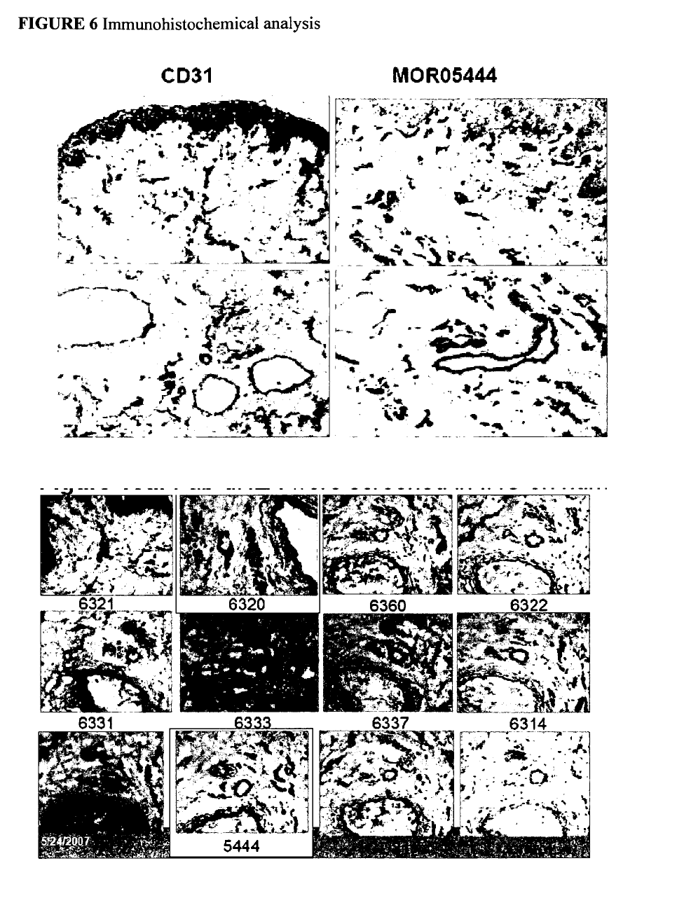

IHC: For IHC HuCAL® anti-Alk1 Fabs and an irrelevant negative control antibody were converted into the bivalent dHLX-format (Plückthun & Pack, 1997). The sixteen Fab-dHLX antibodies were screened on human foreskins sections and human breast carcinoma for positive IHC vessel staining. Two Fab fragments, MOR05444 (FIG. 6) and MOR05377 (not shown) provided specific staining of human blood vessels.

5 μm cryo sections from human breast carcinoma were cut with a Leica CM3050 cryostat. Sections were air-dried for 30 minutes to 1 hour and fixed in ice-cold methanol for 10 minutes and washed with PBS. For the detection of the dHLX-format a mouse anti-His antibody (Dianova) in combination with the Envision Kit (DAKO) was used.

FIG. 6 shows pictures of the respective IHC staining of human foreskin. Endothelial specific staining can be observed with some of the Alk1-specific binders of the present invention (e.g. MOR05444), with a similar pattern as an endothelial specific anti-CD31 antibody, but not in control staining

### Example 14

### Proliferation Assay

### Cells and Culture-Conditions

HUVEC (Human Umbilical Vein Endothelial Cells) and HMVEC-Dly (human lymphatic dermal microvascular endothelial cells) were cultivated in Endothelial cell basal medium (Promocell Cat#C-22211)+supplement pack (2% FCS) (Promocell, Cat#C-39211) (growth medium) on collagen (3% Collagen/PBS) coated flasks.

MVEC Microvascular endothelial cells were cultivated in EBM-2 medium (Cambrex Cat#CC-3156)+Clonetics EGM-2 Single Quots (2% FCS) (Cat#CC-4176) (growth medium) on collagen (3% Collagen/PBS) coated flasks.

### Proliferation Assay

2000 cells of each cell line (HMVEC/HMVEC-Dly/HMEC) were seeded in 100 μl growth medium on collagen (3% Collagen/PBS) coated 96-well (CulturPlate-96, flat bottom, white). After 24 hours, medium was removed and antibodies in 100 μl minimal medium (MM; Promocell Enothelial cell basal medium (Cat#C-22211)+0.2% FCS for HMVEC and HMVEC-Dly; Clonetics EBM-2 (Cambrex, Cat#CC-3156)+0.2% FCS for MVEC) were added. After 72 h incubation the cell number was determined by adding 100 μl CellTiter Glo-Mix (Promega, Cat#G7571, Lot#210907) direct into the medium. Measurement of resulting luminescence occurred after 5 min incubation.

### Results

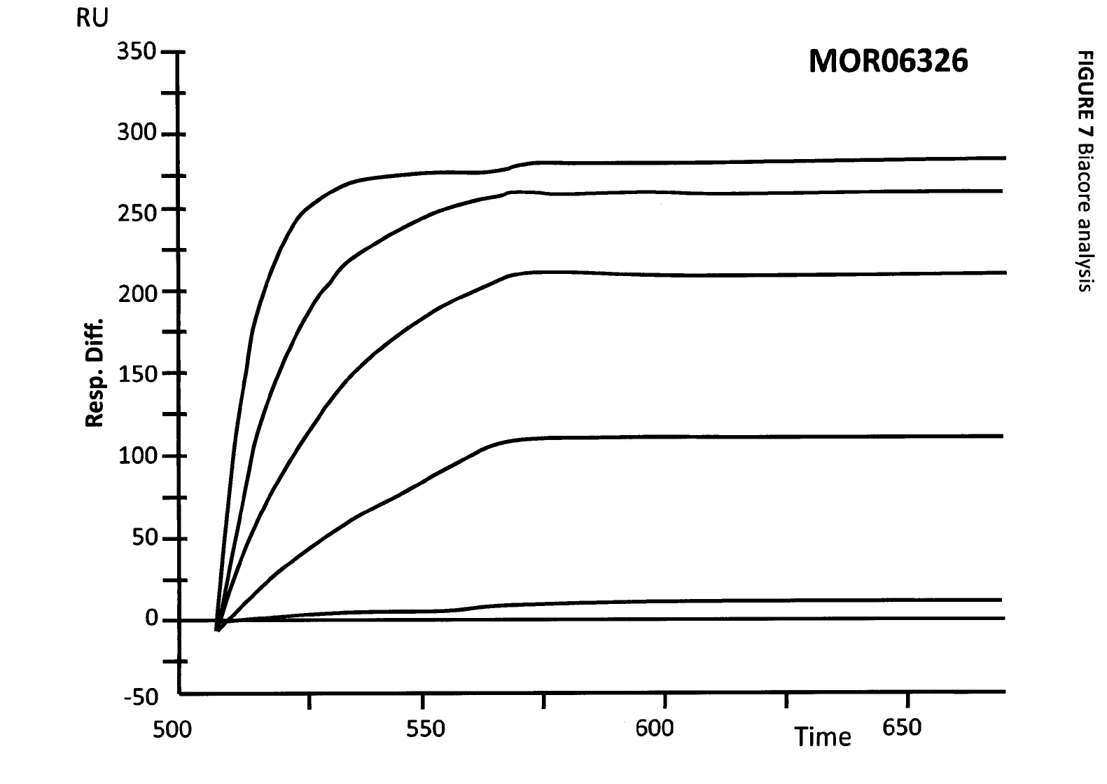

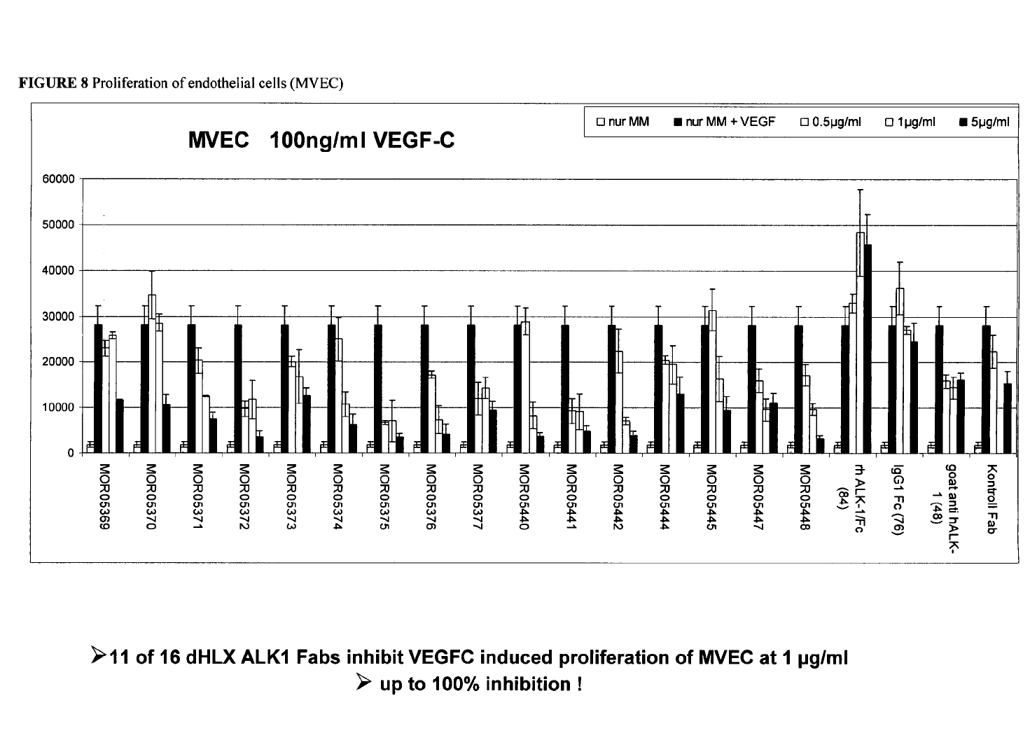

Results of this experiment are depicted in FIGS. 8, 9 and 10. FIG. 8 demostrates that Fab-dHLX antibodies of the present invention can inhibit VEGF-C induced proliferation of MVEC cells. The majority of the antibodies tested inhibit VEGF-C induced proliferation of MVEC cells at a concentration as low as 1 μg/ml, and the inhibiton of proliferation is higher than 90%.

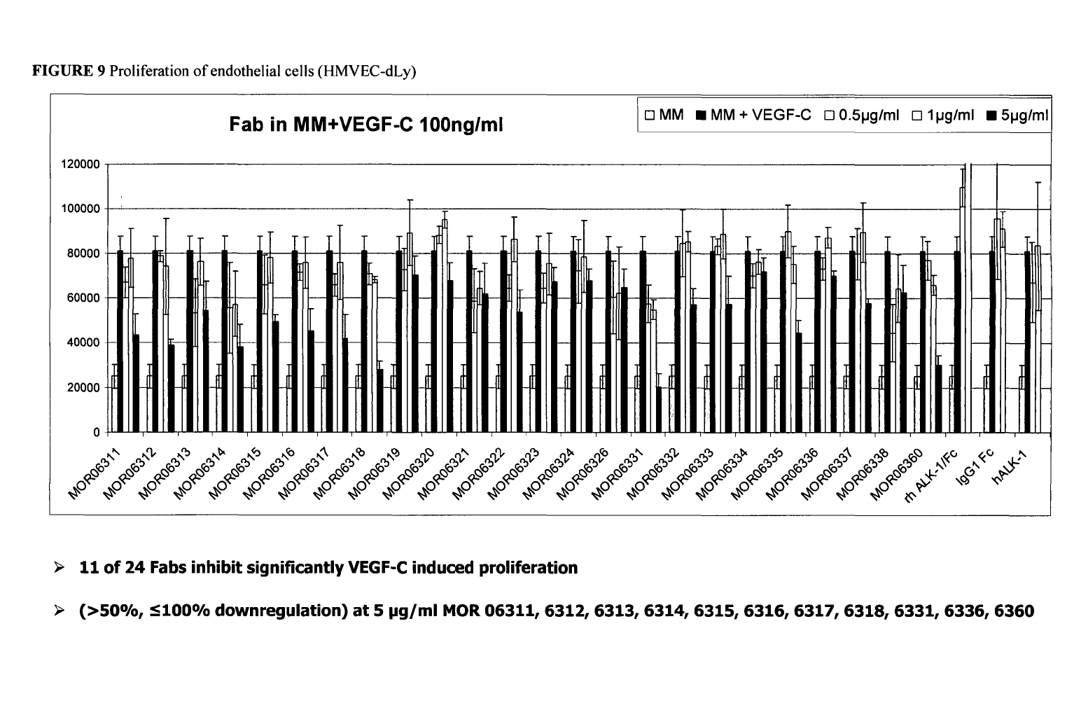

FIG. 9 demostrates that antibodies of the present invention can inhibit VEGF-C induced proliferation of HMVEC cells. Numerous of the antibodies inhibited VEGF-C induced proliferation of HMVEC cells by more than 50% at a concentration of 5 μg/ml.

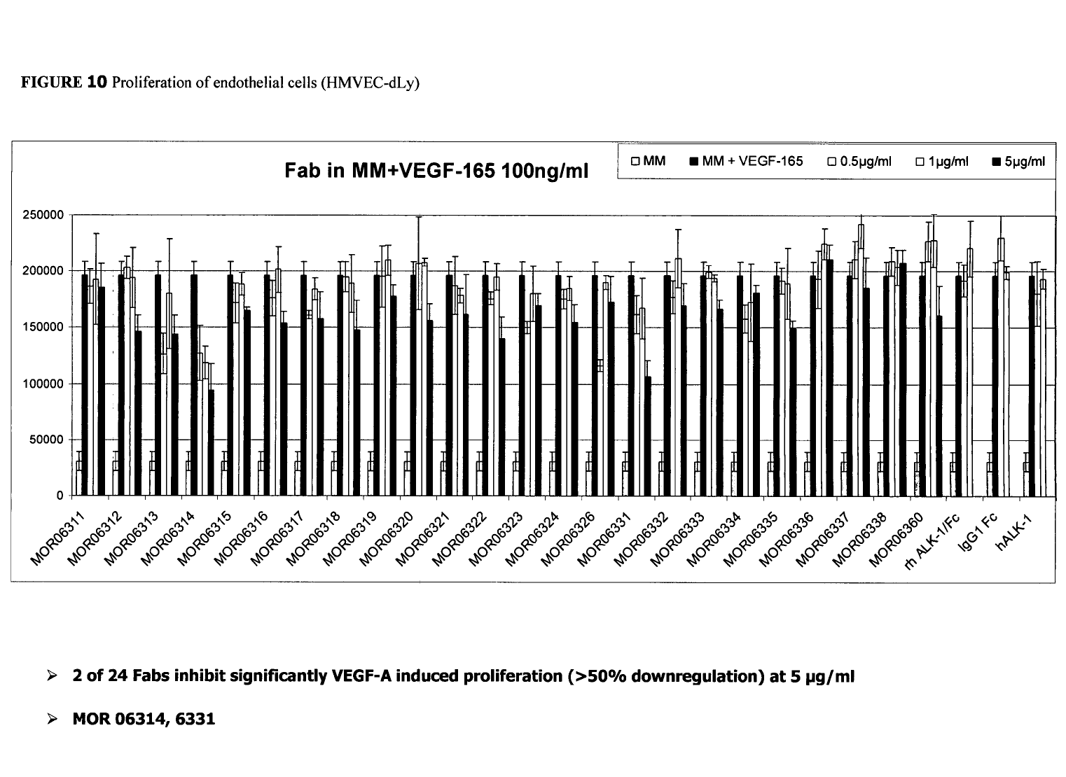

FIG. 10 demostrates that antibodies of the present invention can inhibit VEGF-A induced proliferation of HMVEC cells. Inhibiton of VEGF-A induced proliferation of HMVEC cells by some antibodies is more than 50% at a concentration of 5 μg/ml.

In summary, this experiment demonstrated the pronounced anti-proliferative properties of the antibodies of the present invention in various experimental assays.

### Example 15

### Inhibition of BMP9 Induced SMAD7 expression by Selected Anti Alk1 Antibodies

10000 cells of HMEC-1 or HDMEC-A were seeded in 100 μl growth medium on collagen (3% Collagen/PBS) coated 96-well (CulturPlate-96, flat bottom, white). After 4 hours, medium was removed and minimal medium (MM; EBM-2+0.01% FCS, LONZA# CC4147) were added. After 17 hours, medium was removed and antibodies in 100 μl minimal medium (MM; EBM-2+0.01% FCS, LONZA # CC4147) were added. After 60 min BMP-9 were added to a final concentration 0.1 ng/ml BMP-9. After 2 hours cells were washed (PBS) and lysed (RTL buffer, Qiagen #1015762)

The mRNA isolation occurred in QIAvac 96 (Qiagen #19504) system by Rneasy 96 Kit (Qiagen # 74181/74182). cDNA sysntesis occurred by Omniscript RT Kit (Qiagen #205113).

The expression analysis was performed in a Roche Lightcycler 480 using 5 μl cDNA and Roche Sonde #69, human Smad7 and Roche Sonde #10, human L32 (refence gene) enzyme master MIX (Roche, Real-time PCR Master Mix #4707494/001) Primer for RT-PCR: For human SMAD7:(AF015261.11|AF015261:EMBL|TRAN00000099182:ASTD|9589635:G DB|HIT000062340:H-InvDB Homo sapiens Smad7 protein mRNA, complete cds).

The primerset: Left position 644-664 seq.: CGATGGATTTTCTCAAACCAA and Right position 699-717 seq.: ATTCGTTCCCCCTGTTTCA were used.

For house keeping gene L32 the primer set: SEQ: left AGGGGTTACGACCCATCAG, right GATGCCGAGAAGGAGATGG were used.

The quantification of RNA levels occurred by the ΔΔCT method.

### Results:

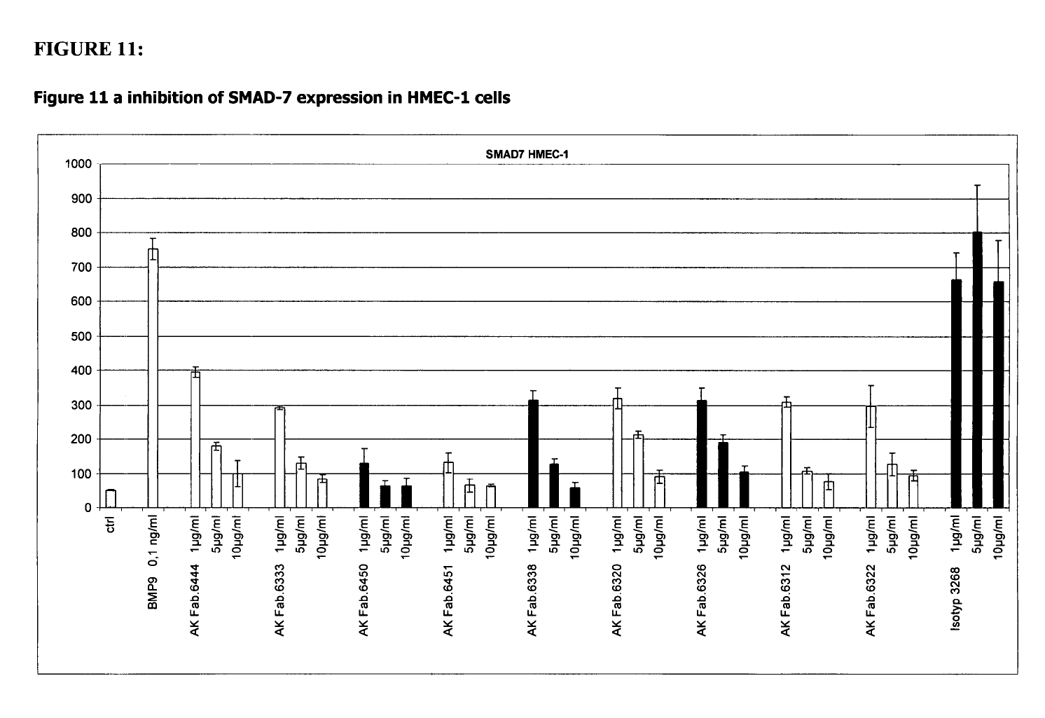

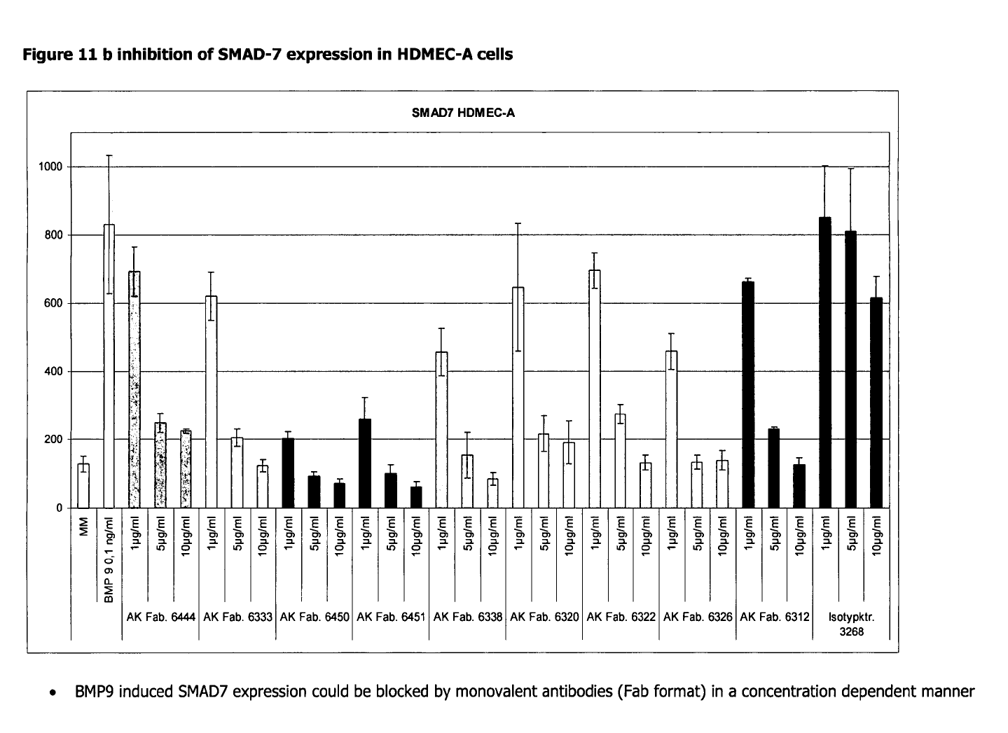

FIGS. 11a and 11b demonstrate that antibodies of the present invention are able to inhibit BMP9 induced expression of SMAD7. In both cell lines tested (HMEC-1 cells and HDMEC-A cells) BMP9 induced expression of SMAD7 could be blocked by monovalent antibodies (Fab format) in a concentration dependent manner. The present invention therefore for the frist time provides antibodies with such properties.

### REFERENCES

Knappik, A., Ge, L., Honegger, A., Pack, P., Fischer, M., Wellnhofer, G., Hoess, A., Wolle, J., Pluckthun, A., and Virnekas, B. (2000). Fully synthetic human combinatorial antibody libraries (HuCAL) based on modular consensus frameworks and CDRs randomized with trinucleotides. J Mol Biol 296, 57-86.

Krebber, A., Bornhauser, S., Burmester, J., Honegger, A., Willuda, J., Bossard, H. R., Plückthun, A. (1997). Reliable cloning of functional antibody variable domains from hybridomas and spleen cell repertoires employing a reengineered phage display system. J. Imm. Meth. 201, 35-55.

Krebs, B., Rauchenberger, R., Reiffert, S., Rothe, C., Tesar, M., Thomassen, E., Cao, M., Dreier, T., Fischer, D., Hoss, A., Inge, L., Knappik, A., Marget, M., Pack, P., Meng, X. Q., Schier, R., Sohlemann, P., Winter, J., Wolle, J., and Kretzschmar, T. (2001). High-throughput generation and engineering of recombinant human antibodies. J Immunol Methods 254, 67-84.

Löhning, C. (2001). Novel methods for displaying (poly)peptides/proteins on bacteriophage particles via disulfide bonds. WO 01/05950.

Plückthun A, and Pack P. (1997) New protein engineering approaches to multivalent and bispecific antibody fragments. Immunotechnology 3(2):83-105.

Rauchenberger R., Borges E., Thomassen-Wolf E., Rom E., Adar R., Yaniv Y., Malka M., Chumakov I., Kotzer S., Resnitzky D., Knappik A., Reiffert S., Prassler J., Jury K., Waldherr D., Bauer S., Kretzschmar T., Yayon A., Rothe C. (2003). Human combinatorial Fab library yielding specific and functional antibodies against the human fibroblast growth factor receptor 3. J Biol Chem. 278(40):38194-205.

David L, Mallet C, Vailhe B, Lamouille S, Feige J J, Bailly S. (2007a) Activin receptor-like kinase 1 inhibits human microvascular endothelial cell migration: Potential roles for JNK and ERK. J Cell Physiol. 213(2):484-9.

David L, Mallet C, Mazerbourg S, Feige J J, Bailly S. (2007b) Identification of BMP9 and BMP10 as functional activators of the orphan activin receptor-like kinase 1(ALK1) in endothelial cells. Blood. 109(5):1953-61

Brown M A, Zhao Q, Baker K A, Naik C, Chen C, Pukac L, Singh M, Tsareva T, Parice Y, Mahoney A, Roschke V, Sanyal I, Choe S. (2005) Crystal structure of BMP-9 and functional interactions with pro-region and receptors. J Biol Chem. 280(26):25111-8

Fernandez-L A, Sanz-Rodriguez F, Blanco F J, Bernabeu C, Botella L M. (2006) Hereditary hemorrhagic telangiectasia, a vascular dysplasia affecting the TGF-beta signaling pathway. Clin Med Res. 4(1):66-78

Goumans M J, Valdimarsdottir G, Itoh S, Lebrin F, Larsson J, Mummery C, Karlsson S, ten Dijke P. (2003) Activin receptor-like kinase (ALK)1 is an antagonistic mediator of lateral TGFbeta/ALK5 signaling. Mol Cell. 12(4):817-28

Haenel C, Satzger M, Ducata DD, Ostendorp R, Brocks B. (2005) Characterization of high-affinity antibodies by electrochemiluminescence-based equilibrium titration. Anal Biochem. 339(1):182-4

Koleva R I, Conley B A, Romero D, Riley K S, Marto J A, Lux A, Vary C P. (2006) Endoglin structure and function: Determinants of endoglin phosphorylation by transforming growth factor-beta receptors. J Biol Chem. 2006 Sep 1;281(35):25110-23.

Knappik,A., Ge, L., Honegger, A., Pack, P., Fischer, M., Wellnhofer, G., Hoess, A., Wolle, J., Pluckthun, A., and Virnekas, B. (2000). Fully synthetic human combinatorial antibody libraries (HuCAL) based on modular consensus frameworks and CDRs randomized with trinucleotides. J Mol Biol 296, 57-86

Lamouille S, Mallet C, Feige J J, Bailly S. (2002) Activin receptor-like kinase 1 is implicated in the maturation phase of angiogenesis. Blood. 100(13):4495-501

Lebrin F, Goumans M J, Jonker L, Carvalho R L, Valdimarsdottir G, Thorikay M, Mummery C, Arthur H M, ten Dijke P. (2004) Endoglin promotes endothelial cell proliferation and TGF-beta/ALK1 signal transduction. EMBO J. 23(20):4018-28

Lebrin F, Deckers M, Bertolino P, Ten Dijke P. (2005) TGF-beta receptor function in the endothelium. Cardiovasc Res. 65(3):599-608

Löhning, C. (2001). Methods for displaying (poly)peptides/proteins on bacteriophage particles via disulfide bonds. U.S. Pat. No. 6,753,136

Mallet C, Vittet D, Feige J J, Bailly S. (2006) TGFbeta1 induces vasculogenesis and inhibits angiogenic sprouting in an embryonic stem cell differentiation model: respective contribution of ALK1 and ALK5. Stem Cells. 24(11):2420-7

Rauchenberger, R., Borges, E., Thomassen-Wolf, E., Rom, E., Adar, R., Yaniv, Y., Malka, M., Chumakov, I., Kotzer, S., Resnitzky, D., Knappik, A., Reiffert, S., Prassler, J., Jury, K., Waldherr, D., Bauer, S., Kretzschmar, T., Yayon, A., and Rothe, C. (2003). Human combinatorial Fab Library yielding specific and functional antibodies against the human fibroblast growth factor receptor 3. J Biol Chem. 278(40):38194-38205

Rondot S, Koch J, Breitling F, Dubel S. (2001) A helper phage to improve single-chain antibody presentation in phage display. Nat Biotechnol. 19(1):75-8

Scharpfenecker M, van Dinther M, Liu Z, van Bezooijen R L, Zhao Q, Pukac L, Löwik C W, ten Dijke P.(2007) BMP-9 signals via ALK1 and inhibits bFGF-induced endothelial cell proliferation and VEGF-stimulated angiogenesis. J Cell Sci. 120(Pt 6):964-72

1. An isolated human or humanized antibody or functional fragment thereof comprising an antigen-binding region that is specific for Alk1 (SEQ ID NO: 193), wherein said antibody or functional fragment thereof posesses one or more of the following properties: a) inhibits proliferation of endothelial cells, b) produces specific staining of tumor blood vessels in IHC, c) inhibits heterodimerization of Alk1 receptor d) inhibits BMP9 stimulated induction of SMAD7 expression in endothelial cells, e) inhibits BMP9 stimulated SMAD 1/5/8 phosphorylation f) possesses a specific affinity (Kd) of 1 nM or below as determined by BIACORE or SET, g) inhibits angiogenesis, h) inhibits tumor growth, i) inhibits endothelial cell migration, j) inhibits smooth muscle cell proliferation, k) inhibits endothelial cell tube formation, and l) induces ADCC in vivo.

2. (canceled)

3. An isolated antigen-binding region according to claim 1, which comprises an H-CDR1 region depicted in SEQ ID NOS: 81-96 or is coded by SEQ ID NOS: 33-48, an H-CDR2 region depicted in SEQ ID NOS: 65-80, or is coded by SEQ ID NOS: 17-32 or 194-261, an H-CDR3 region depicted in SEQ ID NOS: 49-64 or is encoded by SEQ ID NOS: 1-16, an L-CDR1 region depicted in SEQ ID NOS: 177-192 or is coded by SEQ ID NOS: 129-144, an L-CDR2 region depicted in in SEQ ID NOS: 161-176 or is coded by SEQ ID NOS: 113-128, and an L-CDR3 region depicted in SEQ ID NOS: 145-160, or is coded by SEQ ID NOS: 97-112 or 262-329.

4. -8. (canceled)

9. An isolated antigen-binding region according to claim 1, which comprises
a sequence having at least 80 percent sequence identity in the H-CDR regions with the H-CDR regions depicted in SEQ ID NOS: 49-96 and/or 228-261.

10. An isolated antigen-binding region according to any of claim 1, which comprises
a sequence having at least 80 percent sequence identity in the L-CDR regions with the L-CDR regions depicted in SEQ ID NOS: 145-192 and 296-329.

11. An isolated antibody according to claim 1, which is an IgG.

12. An isolated antibody to according to claim 11, which is an IgG1, IgG4 or IgG4-Pro.

13. An antibody or functional fragment thereof according to claim 1, wherein the antibody or functional fragment thereof competes with an antibody that is specific for an epitope comprising one or more amino acid residues of amino acid residues 20-118 of Alk1 (SEQ ID NO: 193), wherein said antibody or functional fragment thereof is able to block the function of Alk1 (SEQ ID NO: 193).

14. (canceled)

15. A nucleic acid sequence encoding an antigen-binding region of an isolated antibody or functional fragment thereof according to claim 1, which comprises
(i) a sequence selected from the group consisting of SEQ ID NOS: 1-48, and 194-227; or
(ii) a nucleic acid sequence that hybridizes under high stringency conditions to the complementary strand of SEQ ID NO: 1-48, or 194-227.

16. A nucleic acid sequence encoding an antigen-binding region of an isolated antibody or functional fragment thereof according to claim 1, which comprises
(i) a sequence selected from the group consisting of SEQ ID NOS: 97-144, and 262-295; or
(ii) a nucleic acid sequence that hybridizes under high stringency conditions to the complementary strand of SEQ ID NO: 97-144, or 262-295, wherein said antibody or functional fragment thereof is specific for an epitope of Alk1.

17. A vector comprising a nucleic acid sequence according to claim 15.

18. An isolated cell comprising a vector according to claim 17.

19. An isolated cell according to claim 18, wherein said cell is bacterial.

20. An isolated cell according to claim 18, wherein said cell is mammalian.

21. A pharmaceutical composition comprising an antibody or functional fragment according to claim 1 and a pharmaceutically acceptable carrier or excipient therefor.

22. A method for treating a disorder or condition associated with the undesired presence of Alk1, comprising administering to a subject in need thereof an effective amount of the pharmaceutical composition according to claim 21.

23. A method according to claim 22, wherein said disorder or condition is cancer.

24. A method according to claim 22, wherein said disorder or condition is macular degeneration.

25. A method for targeting AIkI+cells in a subject or a cell sample, comprising the step of allowing said Alk1+cells to be contacted with an antibody or functional fragment thereof according to claim 1.

26. A method of using an epitope of Alk1 for isolating a human or humanized antibody or functional fragment thereof, comprising the steps of: a) contacting said an epitope of Alk1 comprising amino acid residues 20-118 (SEQ ID NO 193) with an antibody library; and b) isolating an antibody or functional fragment thereof comprising an antigen-binding region that is specific for said epitope.

27. -29. (canceled)

30. A kit comprising an isolated epitope of Alk1 comprising one or more amino acids of amino acids 20-118 of and an antibody library and instructions for use.

31. A human antibody according to claim 1, comprising a synthetic human antibody.

32. An isolated antigen-binding region comprising at least 80% sequence identity to an antigen-binding region according to claim 3.
